SEQUENCE LISTING (14 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 30 aa, Homo sapiens
KAEFHRWSSYMVHWKNQFHDYSKQDRCSDL

SEQ ID NO: 3 - protein, 14 aa, Homo sapiens
AEFHRWSSYMVHWK

SEQ ID NO: 4 - protein, 7 aa, Homo sapiens
SYMVHWK

SEQ ID NO: 5 - protein, 8 aa, Homo sapiens
SSYMVHWK

SEQ ID NO: 6 - protein, 9 aa, Homo sapiens
WSSYMVHWK

SEQ ID NO: 7 - protein, 10 aa, Homo sapiens
RWSSYMVHWK

SEQ ID NO: 8 - protein, 11 aa, Homo sapiens
HRWSSYMVHWK

SEQ ID NO: 9 - protein, 12 aa, Homo sapiens
FHRWSSYMVHWK

SEQ ID NO: 10 - protein, 13 aa, Homo sapiens
EFHRWSSYMVHWK

SEQ ID NO: 11 - protein, 7 aa, Homo sapiens
SYMVHWK

SEQ ID NO: 12 - protein, 4 aa, Homo sapiens
VHWK

SEQ ID NO: 13 - protein, 15 aa, Homo sapiens
NQFDHYSKQDRCSDL

SEQ ID NO: 14 - protein, 42 aa, Homo sapiens [sequence omitted]

(19) United States
(12) Patent Application Publication
(10) Pub. No.: US 2026/0251650 A1
(43) Pub. Date: Aug. 27, 2026

(54) LATERAL FLOW DEVICE FOR DIAGNOSING ALZHEIMER'S DISEASE USING THE T14 PEPTIDE

(71) Applicant: Neuro-Bio Ltd, Abingdon, Oxfordshire (GB)

(72) Inventors: Susan GREENFIELD, Abingdon, Oxfordshire (GB); Sara Garcia-Rates, Abingdon, Oxfordshire (GB)

(21) Appl. No.: 19/109,556

(22) PCT Filed: Sep. 1, 2023
(86) PCT No.: PCT/GB2023/052270

§ 371 Date: Mar. 6, 2025

(30) Foreign Application Priority Data
Sep. 8, 2022 (GB) 2213096.7
Sep. 8, 2022 (WO) PCT/GB2022/052280

(51) Int. Cl. G01N 33/543; G01N 33/68

(52) U.S. Cl. CPC G01N 33/54387; G01N 33/6896; G01N 2333/47; G01N 2800/28; G01N 2800/2821; G01N 2800/52; G01N 2800/56

(57) ABSTRACT

The invention relates to neurodegenerative disorders, and the diagnosis and/or prognosis of neurodegenerative disorders in a test subject using a lateral flow test, or the like. The invention also relates to detecting diagnostic and prognostic biomarkers in a range of various patient sample types for diagnosing and/or prognosing neurodegenerative disorders, such as Alzheimer's disease. The invention further provides biomarker detection methods, and apparatus and apparatuses for diagnosing and prognosing neurodegenerative disorders, and methods of treating patients diagnosed or prognosed with a neurodegenerative disorder. The invention also extends to detection of biomarkers and/or screening in pre-symptomatic subjects, for early diagnosis, to enable disease prevention or intervention.

The invention relates to neurodegenerative disorders, and particularly, although not exclusively, to the diagnosis and/or prognosis of neurodegenerative disorders in a test subject using a lateral flow test, or the like. The invention also relates to detecting diagnostic and prognostic biomarkers in a range of various patient sample types for diagnosing and/or prognosing neurodegenerative disorders, such as Alzheimer's disease. The invention further provides biomarker detection methods, and apparatus and apparatuses for diagnosing and prognosing neurodegenerative disorders, and methods of treating patients diagnosed or prognosed with a neurodegenerative disorder. The invention also extends to detection of biomarkers and/or screening in pre-symptomatic subjects, for early diagnosis, to enable disease prevention or intervention.

Neurodegenerative disorders, such as Alzheimer's disease (AD), Parkinson's disease, Huntington's disease or Motor Neurone disease, are some of the biggest socioeconomic burdens in the world with a growing worldwide incidence rate of almost 10 million new cases of dementia each year. Since both the incidence and prevalence of AD increases with age, the number of patients is growing significantly with an aging population. In 2015, there were over 46 million people living with dementia with an estimated socioeconomic cost of $800 billion annually and it is expected that the number of patients will rise to over 130 million by 2050 costing society over $2 trillion annually. AD was recently announced as the UK's leading cause of death in the over 65's, and in the US it is now the 6th leading cause of death across all ages.

At present, there exists no single test that can be carried out to diagnose Alzheimer's disease, or to prognose cognitive decline (e.g. Braak stages IV, V, or VI). It is also not currently possible to diagnose pre-symptomatic AD (e.g. Braak stages I, II or III). Clinical classification of AD relies on a combination of subjective reporting, medical history evaluation, cognitive function tests, and costly brain imaging scans with no true classification possible until a post-mortem examination of the brain can be conducted. All of these tests require the involvement of expert medical practitioners, but are still of variable accuracy. They also take time, and are, therefore, slow and expensive.

There is, therefore, a need to provide improved methods, apparatuses and/or kits for diagnosing disease in subjects suspected of having a neurodegenerative disorder (e.g. Braak stages I, II or III), and especially Alzheimer's disease, as well as for prognosing disease aetiology or progression in subjects already diagnosed with the condition (e.g. Braak stages IV, V, or VI). There is also a need to provide methods and apparatuses for screening, and detecting the risk of developing a neurodegenerative disorder in subjects who are pre-symptomatic for such conditions, or suspected of being so (e.g. Braak stages I or II). Ideally, such improved methods and apparatuses are quick, quantitative (or at least semi-quantitative or qualitative), accurate, painless and cheap.

The inventors have continued their previous research in this area, and have focused on the toxic peptide, “T14”, which is derived from the C-terminus of acetylcholinesterase (AChE), and which is present as a naturally occurring bioactive molecule in brain tissue. WO 2016/156803 describes an antibody raised against the AChE-derived peptide (T14) peptide, and it has previously been shown that T14 found in cerebrospinal fluid (CSF) or in venous blood samples can act as a robust biomarker for diagnosing neurodegenerative disorders, such as Alzheimer's disease.

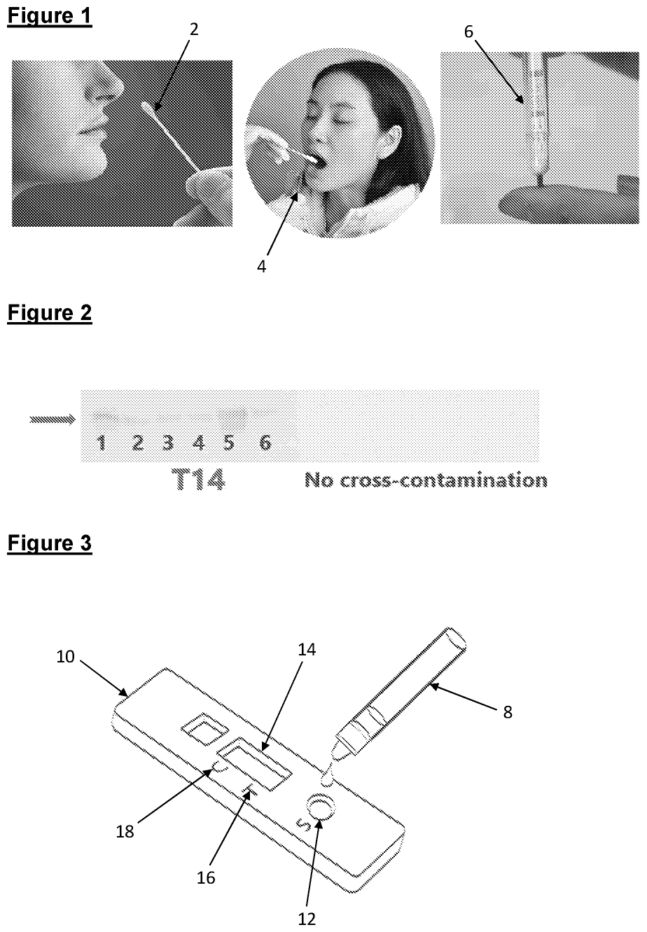

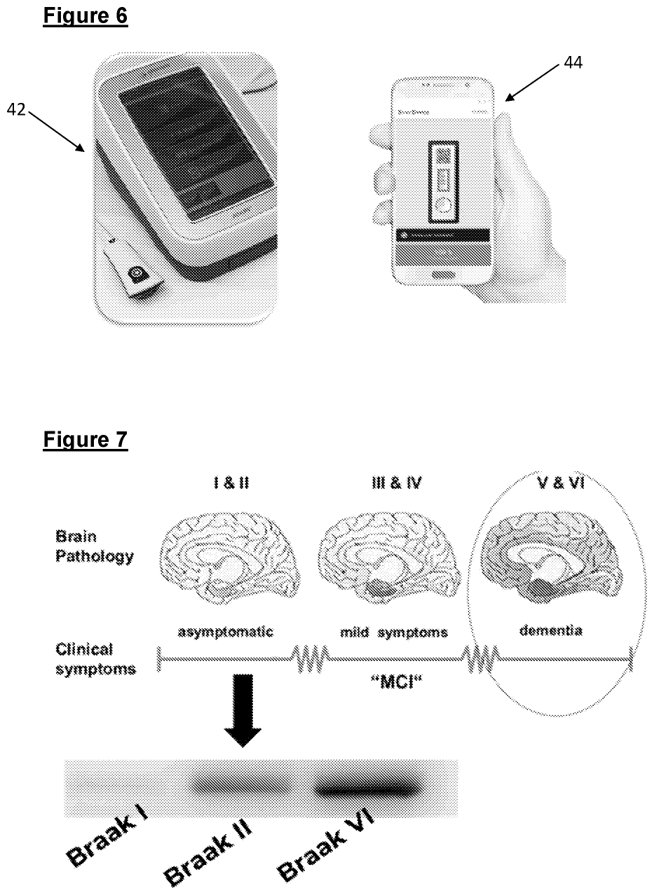

However, as discussed in the Examples and as shown in FIG. 7, the inventors have now surprisingly found that T14 peptide levels are not only elevated in late-stage Alzheimer's disease patients (e.g. Braak stage V or VI), but they are also elevated in the brain at a much earlier stage of neurodegeneration, i.e. at the pre-symptomatic stage of Alzheimer's disease (e.g. Braak stages I, II or III). This was not at all expected. Moreover, as shown in FIG. 2, the inventors have also unexpectedly found that the T14 biomarker is not only detectable in CSF and venous blood draws, but that it is also present in very small, but still detectable, concentrations in various peripheral tissues, such as saliva, nasal discharge and pin-prick blood. Such peripheral tissue types can be readily analysed using a lateral flow test (LFT), which, unlike CSF and venous/arterial blood (as shown in the inventors' previous studies), are painless, socially acceptable by the general public, low cost, can be used frequently (even daily or weekly), and provide very fast and accurate results.

Thus, the inventors believe that, in combination, these data form the basis for a first-in-class reliable diagnostic or prognostic screen for neurodegenerative disorders, as well as for diagnosing pre-symptomatic neurodegenerative disorders, such as early onset Alzheimer's disease.

Thus, in a first aspect of the invention, there is provided a lateral flow method of diagnosing or prognosing a neurodegenerative disorder in a subject, the method comprising using lateral flow to detect, in a sample obtained from a test subject, a peptide comprising or consisting of SEQ ID No: 3 (T14), or a variant or fragment thereof, wherein detecting the presence of the peptide comprising or consisting of SEQ ID No: 3, or a variant or fragment thereof in the sample indicates that the test subject has a neurodegenerative disorder, or a pre-disposition thereto, or a negative prognosis thereof.

In a second aspect of the invention, there is provided a lateral flow apparatus, for diagnosing or prognosing neurodegenerative disorder in a subject, the apparatus comprising a lateral flow support for detecting, in a sample obtained from a test subject, a peptide comprising or consisting of SEQ ID No: 3 (T14), or a variant or fragment thereof, wherein detection of the peptide comprising or consisting of SEQ ID No: 3 (T14), or a variant or fragment thereof corresponds to the subject having a neurodegenerative disorder, or a pre-disposition thereto, or a negative prognosis thereof.

In a third aspect, there is provided a method of treating a subject having or suspected of having a neurodegenerative disorder, pre-symptomatic neurodegenerative disorder and/or suffering from cognitive decline, the method comprising:
(a) using lateral flow to detect, in a sample obtained from a test subject, a peptide comprising or consisting of SEQ ID No: 3 (T14), or a variant or fragment thereof, wherein detecting the presence of the peptide comprising or consisting of SEQ ID No: 3, or a variant or fragment thereof in the sample indicates that the test subject has a neurodegenerative disorder, or a pre-disposition thereto or a negative prognosis thereof; and
(b) administering, or having administered, to the subject, a therapeutic agent that prevents, reduces or delays neurodegeneration and/or cognitive decline.

The inventors believe that they are the first to have developed a method of detecting the T14 peptide in a patient sample using a lateral flow test.

Accordingly, in a fourth aspect, there is provided a method of detecting a peptide comprising or consisting of SEQ ID No: 3 (T14), or a variant or fragment thereof, in a sample obtained from a test subject, the method comprising using lateral flow to detect, in the sample obtained from a test subject, a peptide comprising or consisting of SEQ ID No: 3 (T14), or a variant or fragment thereof.

Because of their much smaller volumes and, therefore, significantly lower concentrations, it was totally unexpected that detectable levels of the T14 peptide (SEQ ID No: 3) would be found in any peripheral bodily samples (such as saliva shown in FIG. 2, nasal discharge or pin prick blood), which are suitable for lateral flow testing. Advantageously, there are various scenarios in which the methods and apparatuses of the invention are of significant value, because they make it possible to accurately and quickly screen, diagnose and prognose neurodegenerative disorder (preferably Alzheimer's disease) using a lateral flow test (LFT).

LFTs are also known in the art as a “rapid test”, “quick test”, or “lateral flow immunoassay (LFIA)”, and so are also covered by this invention.

Firstly, for people who are already confirmed as suffering from Alzheimer's disease, there is a huge value in a rapid and accurate test, which could be used to actively monitor the aetiology or progression of their condition. In addition, having such a test could then be used to influence a more accurate dosage of a therapeutic drug being administered to the patient, and also result in a better patient outcome. Preferably, therefore, the methods and apparatuses are used as prognostic methods or prognostic apparatuses for prognosing the progression of neurodegenerative disorder.

Secondly, there is considerable value in an accurate and fast diagnostic test for use on people who are suspected of having Alzheimer's disease, but are yet to be diagnosed. Such a test would be significantly advantageous compared with the much higher cost, frequency and efficacy of the cognitive testing methods which are currently deployed, e.g. after patient referral to a memory clinic. Accordingly, preferably the methods and apparatuses of the invention are diagnostic methods or diagnostic apparatuses for diagnosing neurodegenerative disorder.

Thirdly, it is clearly very useful to have a convenient test available for people who are not currently suspected of having Alzheimer's, i.e. they are young and/or pre-symptomatic. There is, therefore, a huge opportunity for the general population to have a pre-symptomatic test that could be conducted in a GP surgery or even at home, irrespective of any pre-disposing factors or symptoms, i.e. part of a general medical check-up. For example, the LFT test could be routinely conducted in a certain age population, or above a certain age, similar to breast cancer or bowel cancer screening.

This pre-test screen may be qualitative in nature, e.g. give a binary result of either “positive” or “negative” with respect to a pre-defined range (or window) of T14 values with respect to the risk of having neurodegenerative disease or a pre-disposition thereto. Then, depending on the result of the pre-test screen, the subject may be referred to a doctor, or other specialist medical practitioner, who could then conduct a more accurate quantitative test, and provide expert medical intervention, as required. The pre-test screen may be self-administered. It could, therefore, filter out those subjects who are significantly outside (i.e. having a “negative” result) of the range of T14 values considered to be at risk of neurodegenerative disease, or a pre-disposition thereto, so that only those subject with a “positive” result in the pre-test screen would be subjected to the qualitative test, under supervision, and then discover if they were indeed in the early stages of neurodegenerative disease—or not. Therefore, preferably the methods and apparatuses are used to diagnose pre-symptomatic conditions of patients who will, or could develop neurodegenerative disorder in the future. Preferably, the method is a diagnostic method comprising diagnosing neurodegenerative disorder, preferably pre-symptomatic conditions in the test subject. The ability for early detection will enable early therapeutic intervention and therefore delay onset of the neurodegenerative disorder, or even disease prevention.

Fourthly, pharmaceutical companies would be able save vast amounts of time and costs in their drug development if an accurate measure of disease progression were readily available, in terms of timescales, smaller sample groups, and other value metrics, because the patient would effectively serve as their own control, as it could be monitored how much they had, or had not, deteriorated since an earlier test (e.g. a day, week or month previously). Therefore, preferably the methods and apparatuses are used in a clinical trial to monitor the activity or efficacy of a drug administered to the test subject, and preferably determine how much the subject has, or has not, deteriorated since an earlier test.

Preferably, the lateral flow apparatuses or methods of the first to fourth aspects are carried out in vitro. It will be appreciated that a lateral flow test is an immunoassay, but is not ELISA. Thus, preferably the peptide SEQ ID No: 3 (T14), or a variant or fragment thereof is not determined using ELISA, i.e. non-ELISA methods and kits/apparatuses.

Preferably, the lateral flow apparatuses or methods of the first to fourth aspects are used to identify the presence or absence of the peptide of SEQ ID No: 3 (T14), or a variant or fragment thereof in the sample, and/or determine the concentration thereof in the sample, preferably the concentration of soluble T14. T14 peptide (SEQ ID No: 3) may be assayed by a number of lateral flow systems that are known to the skilled technician. Lateral flow is a form of immunoassay which is employed to detect T14 peptide or determine T14 levels. Preferably, the methods (or assays) or apparatuses are adapted to detect the presence and/or absence of T14 in the sample. The lateral flow apparatuses or methods may comprise the use of a positive control and/or a negative control against which the assay may be compared.

It is especially preferred that the methods or apparatuses of the invention comprise detection of soluble T14 (SEQ ID No: 3, or a variant or fragment thereof). In one embodiment, the concentration of a soluble peptide comprising or consisting of SEQ ID No: 3, or a variant or fragment thereof, is determined. Preferably, the methods or apparatuses comprise means for determining, in the sample obtained from the test subject, the concentration of SEQ ID No: 3, or a variant or fragment thereof. The means for determining, in the sample obtained from the test subject, the concentration of SEQ ID No: 3, or a variant or fragment thereof may comprise an anti-T14 antibody or antigen-binding fragment thereof, i.e. a T14-neutralising antibody. The antibody or antigen-binding fragment thereof may be polyclonal or monoclonal. The antibody or antigen-binding fragment thereof may be generated in a rabbit, mouse or rat.

Embodiments of the lateral flow method and apparatus of the first to fourth aspects are shown in FIGS. 1, and 3-6. Preferably, the methods of apparatus comprise the use of sample extraction means for obtaining the same from the test subject. For example, the sample extraction means may comprise a nasal swab, a mouth swab or pin-prick blood extraction means. Each of these tools for taking a sample for the lateral flow is shown in FIG. 1.

Once the sample has been obtained from the subject, it may then be inserted into a suitable sample collection container. The container preferably comprises a buffering solution. In one embodiment, the buffering solution may comprise 0.5 M Tris HCl, pH 6.8, 10% glycerol, 2% (w/v) sodium dodecyl sulphate, 5% (v/v) 2-b-mercaptoethanol, 0.05% bromophenol blue. Preferably, the sample is mixed with the buffering solution until any T14 peptide therein is suspended.

Preferably, the apparatus comprises an LFT cartridge which has a sample aperture into which buffered sample solution is delivered. Preferably, the cartridge comprises an aperture or window through which a test line and a control line may be viewed.

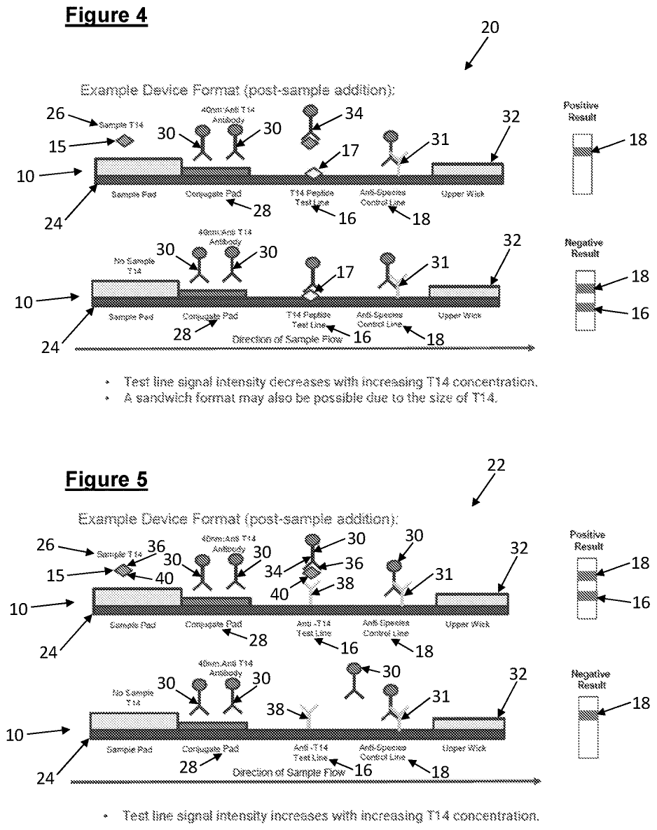

FIGS. 4 and 5 show two different preferred embodiments of lateral flow apparatus and methods of the invention. Advantageously, the LFT assays described herein is qualitative, semi-quantitative or quantitative. In one embodiment, the apparatus or methods preferably comprise an inhibition lateral flow test, which is shown in FIG. 4. In another embodiment, the apparatus or methods preferably comprise a sandwich lateral flow test, which is shown in FIG. 5.

Preferably, the LFT cartridge comprises a sample pad onto which the sample is placed. Preferably, the LFT cartridge comprises anti-T14 antibodies, which are preferably disposed on a conjugate pad. For example, the anti-T14 antibodies can be as described in WO 2016/156803. Preferably the conjugate pad is disposed substantially adjacent to the sample pad. Preferably, the anti-T14 antibodies are labelled with a labelling moiety. For example, the labelling moiety may comprise a nanoparticle, preferably a gold nanoparticle. The average diameter of the nanoparticle may be between 10 nm and 65 nm, or between 20 nm and 60 nm or between 30 and 50 nm.

Preferably, the LFT cartridge comprises a fixed T14 peptide test line comprising T14 peptide attached thereto. Preferably, the LFT cartridge comprises a control line comprising an anti-species labelled antibody, which is not specific for T14. For example, the anti-species labelled antibody may comprise a goat anti-chicken antibody or a goat anti-rabbit antibody. For example, the anti-species labelled antibody may comprise a goat anti-chicken IgY polyclonal antibody or a goat anti-rabbit IgY polyclonal antibody. Preferably, the control line is laterally spaced apart from the fixed T14 peptide test line, and preferably distal from the sample pad, and conjugate pad. Preferably, the LFT cartridge comprises a wick disposed at or towards the opposite end of the cartridge from the sample pad, and is configured to draw the sample solution laterally across the cartridge.

As illustrated in the upper part of FIG. 4 (showing a positive result for T14 detection using an inhibition lateral flow test), when the sample comprises sample T14 peptide, and it flows laterally over the conjugate pad harbouring the anti-T14 antibodies, it is preferably captured by the anti-T14 antibody creating a conjugate of T14-antibody. The T14-antibody conjugate is preferably unable to bind to the fixed T14 peptide that is attached to the test line. However, preferably anti-T14 antibody that is not bound to sample T14 peptide in the sample may flow and bind to the anti-species labelled antibody attached to the control line. It will be appreciated that the sample T14 arrests the anti-T14 antibody which cannot bind the fixed T14 peptide attached to the cartridge. Accordingly, preferably a single line develops on the cartridge as a positive result, as shown to the upper right of FIG. 4.

As illustrated in the lower part of FIG. 4 (showing a negative result for T14 detection using an inhibition lateral flow test), when the sample does not comprise sample T14 peptide, the sample preferably flows laterally over the conjugate pad harbouring the anti-T14 antibodies. As there is no sample T14 present to be captured by the anti-T14 antibody, preferably no conjugate of T14-antibody is created. Accordingly, all unbound anti-T14 antibody preferably flows and binds to the LFT fixed T14 peptide that is attached to the test line, and preferably to the anti-species antibody control attached to the control line. Accordingly, two spaced apart lines preferably develop on the cartridge as a negative result, as shown to the lower right of FIG. 4.

Referring now to FIG. 5, the inventors have also developed a sandwich (i.e. two antibody) assay format due to the size of the T14 peptide. Preferably, the methods or apparatus comprise a first antibody which binds to a first epitope disposed in one region of the T14 peptide (e.g. at or towards the N-terminus), and a second antibody which binds to a second epitope on the T14 peptide, which is spaced apart from the first epitope (e.g. at or towards the C-terminus). Preferably, the first and second epitopes are different amino acids on the T14 peptide.

Preferably, the LFT cartridge comprises a sample pad onto which the sample is placed. Preferably, the LFT cartridge comprises first anti-T14 antibodies, which are preferably disposed on a conjugate pad. For example, the anti-T14 antibodies can be as described in WO 2016/156803. The first anti-T14 antibodies preferably bind to the first epitope on the T14 peptide, such as the C-terminal residues of T14. Preferably the conjugate pad is disposed substantially adjacent to the sample pad. Preferably, the anti-T14 antibodies are labelled with a labelling moiety. For example, the labelling moiety may comprise a nanoparticle, preferably a gold nanoparticle.

Preferably, the LFT cartridge comprises a T14 peptide test line comprising second anti-T14 antibodies. Preferably, the second anti-T14 antibodies bind to a second epitope on the T14 peptide. For example, the second anti-T14 antibodies can bind to the N-terminus of T14.

Preferably, the LFT cartridge comprises a control line comprising an anti-species labelled antibody, which is not specific for T14. For example, the anti-species labelled antibody may comprise a goat anti-chicken antibody or a goat anti-rabbit antibody. For example, the anti-species labelled antibody may comprise a goat anti-chicken or anti-rabbit IgY polyclonal antibody. Preferably, the control line is laterally spaced apart from the fixed T14 peptide test line, and preferably distal from the sample pad, and conjugate pad. Preferably, the LFT cartridge comprises a wick disposed at or towards the opposite end of the cartridge from the sample pad, and is configured to draw the sample solution laterally across the cartridge.

As illustrated in the upper part of FIG. 5 (showing a positive result for T14 detection using a sandwich lateral flow test), when the sample comprises T14 peptide, and it flows laterally over the conjugate pad harbouring the first anti-T14 antibodies, it is preferably captured by the anti-T14 antibody thereby creating a conjugate of T14-antibody. Preferably, the T14-antibody conjugate is itself captured by the second anti-T14 antibody which is attached to the cartridge along the test line (16). This is possible because the second epitope on T14 peptide is still exposed in the conjugate. Accordingly, a first line preferably develops on the cartridge signifying antibody binding at the T14 test line. Preferably, any additional anti-T14 antibody that has not bound to T14 peptide in the sample flows and preferably binds to the anti-species control affixed to the control line. Advantageously, unlike in the inhibition LFT assay described above, the sandwich assay, because the cartridge does not include any LFT fixed T14, the sandwich assay using the first and second anti-T14 antibodies is a direct detection of the sample T14 present in the sample. Accordingly, a second line develops on the cartridge as a positive result, as shown to the upper right of FIG. 5.

As illustrated in the lower part of FIG. 5 (showing a negative result for T14 detection using a sandwich lateral flow test), when the sample does not comprise T14 peptide, and it preferably flows laterally over the conjugate pad harbouring the first anti-T14 antibodies, there is no T14 present to be captured by the anti-T14 antibodies, and so preferably no conjugate of T14-antibody is created. Preferably, all unbound first anti-T14 antibody flows over and cannot bind to the second T14 antibody attached to the cartridge. The T14 antibody preferably flows to and binds the anti-species control affixed to the control line, and preferably creates one line on the cartridge (10) as a negative result, as shown to the lower right of FIG. 5.

It will be appreciated from the foregoing that the lateral flow tests described above provide a fast and convenient means for detecting the presence or absence of the T14 peptide or variant of fragment thereof in the sample taken from the subject.

Advantageously, the presence of the control line ensures that the method is robust and valid, and the presence or absence of the test line informs the subject of the presence or absence of T14 peptide or variant or fragment thereof in their sample. Visually assessing the presence or absence of the test line provides a qualitative diagnostic or prognostic test, and visually determining the relative thickness (or darkness in colour) of the test line provides a useful semi-quantitative measure. As such, the use of a lateral flow test for screening for T14 is highly valuable, and could be carried out by a subject on their own (i.e. not in the presence of qualified medical staff) at home.

However, achieving accurate and fully quantitative assessments from merely looking at an LFT result is challenging. Accordingly, preferably the methods or apparatus may comprise means for quantitatively measuring the results of the LFT cartridge. In one embodiment, the lateral flow cartridge may be connected to a reader, which runs software for detecting the test line and control line and then, based on the concentrations/values of the test line and control line, accurately calculates the concentration of T14 peptide in the sample.

In another embodiment, the cartridge may be connected to a computer, tablet or smartphone running software for detecting the test line and control line, and then, based on the concentrations/values of the test line and control line, calculate the concentration of T14 peptide in the sample. Alternatively, a user may take a photograph of the LFT cartridge and the software determines the levels of T14 peptide in the sample based on the strength or intensity of signal in the test line and control line.

Preferably, the sample comprises a biological sample. The sample may be any biological material that is obtainable from the subject from which SEQ ID No: 3 (T14), or a variant or fragment thereof is obtainable.

The sample may be nasal discharge or fluid, saliva, blood, venous blood, arterial blood, blood plasma, blood serum, capillary blood, non-venous blood, non-arterial blood, pin-prick blood, spinal fluid, urine, sweat, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof.

Preferably, the sample does not comprise blood, most preferably not venous or arterial blood. Preferably, the sample does not comprise saliva.

Preferably, however, the sample comprises a peripheral tissue sample. Preferably, the sample comprises nasal discharge or nasal fluid, saliva, capillary blood or pin-prick blood.

The apparatus may comprise a sample collection container for receiving the extracted sample obtained from the subject. Blood samples may be assayed for T14 levels immediately. Alternatively, the blood sample may be stored at low temperatures, for example in a fridge or even frozen before the T14 assay is conducted. Detection of T14 peptide may be carried out on whole blood taken from a blood sample. Preferably, however, the blood sample comprises blood serum taken. Preferably, the blood sample comprises blood plasma.

The blood may be further processed before the T14 assay is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting. Alternatively, the blood sample may be centrifuged or filtered to prepare a plasma or serum fraction, which may be used for analysis. Hence, it is preferred that the T14 is analysed or assayed in a blood plasma or a blood serum sample. It is especially preferred that T14 concentration is measured in vitro from a blood serum sample or a plasma sample taken from the subject.

Most preferably, the sample comprises a capillary blood sample, preferably a pin-prick blood sample. Most preferably, the sample comprises a saliva sample. Most preferably, the sample comprises a nasal discharge or nasal fluid sample. Advantageously, any of the above samples can be used in an LFT methods or apparatus. However, it is also possible to detect the T14 peptide in these samples (and especially nasal discharge/fluid, and pin-prick blood samples) by non-LFT means.

Thus, in a fifth aspect, there is provided a method of diagnosing or prognosing a neurodegenerative disorder in a subject, the method comprising detecting, in a nasal discharge, nasal fluid, or a pin-prick blood sample obtained from a test subject, a peptide comprising or consisting of SEQ ID No: 3 (T14), or a variant or fragment thereof, wherein detecting the presence of the peptide comprising or consisting of SEQ ID No: 3, or a variant or fragment thereof in the sample indicates that the test subject has a neurodegenerative disorder, or a pre-disposition thereto or a negative prognosis thereof.

In a sixth aspect of the invention, there is provided a neurodegenerative disorder diagnostic or prognostic apparatus, for diagnosing or prognosing neurodegenerative disorder in a subject, the apparatus comprising means for detecting, in a nasal discharge, nasal fluid, or a pin-prick blood sample obtained from a test subject, a peptide comprising or consisting of SEQ ID No: 3 (T14), or a variant or fragment thereof, wherein detection of the peptide comprising or consisting of SEQ ID No: 3 (T14), or a variant or fragment thereof corresponds to the subject having a neurodegenerative disorder, or a pre-disposition thereto or a negative prognosis thereof.

In a seventh aspect, there is provided a method of treating a subject having or suspected of having a neurodegenerative disorder, pre-symptomatic neurodegenerative disorder and/or suffering from cognitive decline, the method comprising:
(a) detecting, in a nasal discharge, nasal fluid, or a pin-prick blood sample obtained from a test subject, a peptide comprising or consisting of SEQ ID No: 3 (T14), or a variant or fragment thereof, wherein detecting the presence of the peptide comprising or consisting of SEQ ID No: 3, or a variant or fragment thereof in the sample indicates that the test subject has neurodegenerative disorder, or a pre-disposition thereto or a negative prognosis thereof; and
(b) administering or having administered, to the subject, a therapeutic agent that prevents, reduces or delays neurodegeneration and/or cognitive decline.

The methods or apparatus of the fifth to seventh aspects may be carried out in vivo, in vitro or ex vivo. However, preferably the methods are carried out in vitro.

Most preferably, the sample comprises nasal discharge or nasal fluid. Most preferably, the sample comprises saliva.

Preferably, the apparatuses or methods of the fifth to seventh aspects are used to identify the presence or absence of T14 (SEQ ID No: 3, or variant or fragment thereof) in the sample, or determine the concentration thereof in the sample, preferably the concentration of soluble T14. The means for determining the T14 concentration may comprise an assay adapted to detect the presence and/or absence of T14 in the sample. The apparatuses or methods may comprise the use of a positive control and/or a negative control against which the assay may be compared.

Although the methods or apparatuses of the first to fourth aspects are comprise the use of lateral flow, for the methods or apparatuses of the fifth to sixth aspects, the T14 peptide (SEQ ID No: 3) may be assayed by a number of ways known to one skilled in the art, and not necessarily lateral flow. For example, preferably an immunoassay is employed to detect T14 peptide or determine T14 levels. However, it will be appreciated that non-immuno based assays may also be employed, for example, labelling a compound having affinity with a ligand of the T14 peptide, and then assaying for the label. T14 peptide may also be determined with Western Blot analysis, which may be used to determine the total protein level of T14 peptide. T14 peptide concentration may therefore be detected by enzyme-linked immunosorbent assay (ELISA), fluorometric assay, chemiluminescent assay, or radioimmunoassay analyses.

An immunoassay (e.g. ELISA of LFT), is most preferably used to detect soluble T14 peptide. Western Blot analysis is most preferably used to detect aggregated T14 peptide. It is especially preferred that the methods and apparatuses and uses of the invention comprise detection of soluble T14 (SEQ ID No: 3).

In one embodiment, the concentration of (i) a soluble peptide comprising or consisting of SEQ ID No: 3, or a variant or fragment thereof, or of (ii) an aggregated peptide comprising or consisting of SEQ ID No: 3, or a variant or fragment thereof, is determined. In a preferred embodiment, however, the concentration of (i) a soluble peptide comprising or consisting of SEQ ID No: 3, or a variant or fragment thereof, and of (ii) an aggregated peptide comprising or consisting of SEQ ID No: 3, or a variant or fragment thereof, is determined. It is preferred that both soluble and aggregated T14 can be detected in combination.

Preferably, however, the apparatuses or methods of the fifth to seventh aspects comprise the use of a lateral flow assay or test as in the apparatuses or methods of the first to fourth aspects.

Preferably, the methods or apparatuses of the fifth to seventh aspects comprise means for determining, in the sample obtained from the test subject, the concentration of SEQ ID No: 3, or a variant or fragment thereof. The means for determining, in the nasal discharge, nasal fluid, or a pin-prick blood sample obtained from the test subject, the concentration of (i) a soluble T14 and/or (ii) an aggregated T14 may comprise an anti-T14 antibody or antigen-binding fragment thereof, i.e. a T14-neutralising antibody. The antibody or antigen-binding fragment thereof may be polyclonal or monoclonal. The antibody or antigen-binding fragment thereof may be generated in a rabbit, mouse or rat.

Any of the methods or apparatuses described herein comprise the use of an anti-T14 immunospecific antibody or antigen-binding fragment thereof. Preferably, the antibody or antigen-binding fragment thereof specifically binds to SEQ ID No: 3. Preferably, the antibody or antigen-binding fragment thereof specifically binds to one or more amino acid in the C-terminus of SEQ ID No: 3. Preferably, the antibody or antigen-binding fragment thereof specifically binds to one or more amino acid in SEQ ID No: 11 (i.e. SYMVHWK, which are the C-terminal amino acids numbers 7-14 of SEQ ID No: 3). Preferably, the antibody or antigen-binding fragment thereof specifically binds to a C-terminal lysine (K) residue in the epitope. The inventors have surprisingly observed that the C-terminal amino acid sequence VHWK in SEQ ID No: 3, which is described herein as SEQ ID No. 12 (i.e. the C-terminal amino acids numbers 11-14 of SEQ ID No. 3), acts as an epitope for the antibody or antigen-binding fragment thereof. Accordingly, more preferably the antibody or antigen-binding fragment thereof specifically binds to one or more amino acid in SEQ ID No. 12. Most preferably, the antibody or antigen-binding fragment thereof specifically binds to SEQ ID No. 12. Hence, it will be appreciated that the epitope to which the antibody binds comprises or consists of SEQ ID No: 12. Thus, the antibody or antigen-binding fragment thereof binds specifically to SEQ ID No: 3, or a fragment or variant thereof, and can be used as or in the T14 peptide detection means.

Preferably, the antibody or antigen-binding fragment thereof does not bind to SEQ ID No: 2 (i.e. T30).

Preferably, the antibody or antigen-binding fragment thereof does not bind to SEQ ID No: 13 (i.e. T15), i.e. NQFDHYSKQDRCSDL.

Preferably, the antibody or antigen-binding fragment thereof does not bind to SEQ ID No: 14 (i.e. β-amyloid (Aβ), i.e.

DAEFRHDSGYEVHHQKLVFFAEDVGSNKGAIIGLMVGGVVIA.

The apparatuses or methods may further comprise the use of a label which may be detected in the assay. The term “label” can mean any moiety that can be attached to the means for determining, in the sample obtained from the test subject, the concentration of T14 peptide, be it soluble T14 and/or aggregated T14.

Moieties can be used, for example, for therapeutic or diagnostic procedures. Therapeutic labels include, for example, moieties that can be attached to the antibody or fragment thereof described herein, and used to monitor the binding of the antibody to the T14 peptide (i.e. SEQ ID No: 3, or fragment or variant thereof). Diagnostic labels include, for example, moieties which can be detected by analytical methods. Analytical methods include, for example, qualitative, semi-quantitative and quantitative procedures. Qualitative analytical methods include, for example, immunohistochemistry and indirect immunofluorescence. Quantitative analytical methods include, for example, immunoaffinity procedures such as radioimmunoassay, ELISA or FACS analysis. Analytical methods also include both in vitro and in vivo imaging procedures. Specific examples of diagnostic labels that can be detected by analytical means include enzymes, radioisotopes, fluorochromes, chemiluminescent markers, and biotin.

Preferably, T14 peptide concentrations may be measured by double-antibody sandwich ELISA, or LFT. The ELISA may comprise using a suitable antibody, for example for coating a microtiter plate (for ELISA) or substrate (for LFT). For example, such a suitable antibody may comprise an anti-T14 peptide antibody described herein (WO 2016/156803). Furthermore, the ELISA may comprise using a suitable antibody for detection. For example, such a suitable antibody may comprise peroxidase-labelled monoclonal mouse anti human T14 peptide antibody. Human T14 peptide, which may be purified from blood plasma of nasal discharge, and which then may be quantified by amino acid analysis, may be used to calibrate a plasma or nasal discharge standard using standard techniques known to the skilled technician. A label can be attached directly to the antibody, or it may be attached to a secondary binding agent that specifically binds T14. Such a secondary binding agent can be, for example, a secondary antibody. A secondary antibody can be either polyclonal or monoclonal, and of human, rodent or chimeric origin.

Pin prick blood samples used in the apparatuses or methods of the fifth to seventh aspects may be assayed for T14 levels immediately. Alternatively, the pin-prick blood sample may be stored at low temperatures, for example in a fridge or even frozen before the T14 assay is conducted. Detection of T14 may be carried out on whole blood obtained from the pin prick. Preferably, however, the pin prick blood sample comprises blood serum. Preferably, the pin prick blood sample comprises blood plasma.

The blood may be further processed before the T14 assay is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting. Alternatively, the blood sample may be centrifuged or filtered to prepare a plasma or serum fraction, which may be used for analysis. Hence, it is preferred that the T14 is analysed or assayed in a blood plasma or a blood serum sample. It is especially preferred that T14 concentration is measured in vitro from a blood serum sample or a plasma sample taken from the subject.

Preferably, the apparatus or method is used to identify the presence or absence of T14 (SEQ ID No: 3, or variant or fragment thereof) in the sample, or determine the concentration thereof in the sample, preferably the concentration of T14. The means for determining the T14 concentration may comprise an assay adapted to detect the presence and/or absence of T14 in the sample. The apparatus or method may comprise the use of a positive control and/or a negative control against which the assay may be compared.

The concentration of the peptide comprising or consisting of SEQ ID No: 3, or a variant or fragment thereof in the sample may be between 0.1 and 1000 ng, or between 0.2 and 750 ng, or between 0.5 and 500 ng per mg protein in the sample. Preferably, the concentration of the peptide comprising or consisting of SEQ ID No: 3, or a variant or fragment thereof in the sample is between 1 and 400 ng, or between 2 and 300 ng, or between 3 and 200 ng per mg protein in the sample. Preferably, the concentration of the peptide comprising or consisting of SEQ ID No: 3, or a variant or fragment thereof in the sample is between 4 and 100 ng, or between 5 and 75 ng, between 10 and 50 ng, or between 20 and 40 ng per mg protein in the sample.

The invention may be used in a method or apparatus, where the subject has, or is suspected of having, a neurodegenerative disease selected from a group consisting of: Alzheimer's disease; Parkinson's disease; Huntington's disease; Motor Neurone disease; Spinocerebellar type 1, type 2, and type 3; Amyotrophic Lateral Sclerosis (ALS); schizophrenia; Lewy-body dementia; and Frontotemporal Dementia.

It is preferred, however, that the invention is used to study or predict cognitive decline in any neurological disorder associated with non-enzymatic function of AChE. Preferably, therefore, the neurodegenerative disease is selected from a group consisting of: Alzheimer's Disease, Parkinson's Disease and Motor Neuron Disease, and preferably Alzheimer's Disease or Parkinson's Disease.

However, it is especially preferred that the methods and apparatuses of invention are used where the subject has, or is suspected of having, Alzheimer's Disease.

Therefore, it will be appreciated that, in a preferred embodiment, the method or apparatus of the invention may comprise the use of lateral flow to detect of SEQ ID No: 3, or a variant or fragment thereof in a sample, which is preferably nasal discharge, nasal fluid or saliva, to diagnose or prognose Alzheimer's Disease.

Examples of suitable therapeutic agents which may be administered to the subject to prevent or treat the neurodegeneration and/or cognitive decline include, but are not limited to, acetylcholinesterase inhibitors, such as Rivastigmine, Galantamine, and Donepezil, and/or N-methyl-D-aspartate (NMDA) antagonists, such as Memantine. Alternative therapies include the use of a cyclic peptide as described in WO2015/004430, a linear peptide as described in WO2015/053601, or a peptidomimetic as described in WO2018/033724, the contents of these three patent applications all being incorporated herein by reference.

Preferably, the subject who is tested is a living subject. The subject may be a vertebrate, mammal, or domestic animal. Most preferably, however, the subject is a human being, who may be male or female. The subject may be a child or adult. The age of the subject may be at least 20, 30, 40, 50, 60, 65 or 70 years old. The subject, however, may be less than 80, 70, 65, 60 or 50 years old.

In one embodiment, the methods and apparatus are used on a subject already diagnosed with a neurodegenerative disorder. Accordingly, the subject is preferably symptomatic and showing signs of cognitive decline or dementia. Thus, the methods and apparatus may be prognostic and monitor disease progression.

In another embodiment, the methods and apparatus are used on a subject who is suspected of having a neurodegenerative disorder. The subject may be symptomatic for a neurodegenerative disorder. Thus, the methods and apparatus may be diagnostic.

In a preferred embodiment, the methods and apparatus are used on a subject who is not suspected of having a neurodegenerative disorder. Preferably, the subject is tested before any symptoms of neurodegeneration, cognitive decline or neurodegenerative disorder are apparent. Preferably, the subject is pre-symptomatic. Preferably, the subject is Braak stage I or II. Thus, the methods and apparatus may be diagnostic.

Therefore, it will be appreciated that, in a preferred embodiment, the method or apparatus of the invention may comprise the use of lateral flow to detect of SEQ ID No: 3, or a variant or fragment thereof in a sample, which is preferably nasal discharge, nasal fluid or saliva, to diagnose or prognose Alzheimer's Disease, most preferably pre-symptomatic AD.

Following on from their previous studies, the inventors continued their research with acetylcholinesterase, and their antibody which exhibits immunospecificity to specific regions in the C-terminus of this enzyme, as described in WO 2016/156803, the contents of which are incorporated herein by reference.

Acetylcholinesterase is a serine protease that hydrolyses acetylcholine, and is well-known to the skilled person. The major form of acetylcholinesterase which is found in the brain is known as tailed acetylcholinesterase (T-AChE). The protein sequence of one embodiment of human tailed acetylcholinesterase (Gen Bank: AAA68151.1) is 614 amino acids in length, and is provided herein as SEQ ID No: 1, as follows:

| [SEQ ID No:1] | |
|---|---|
| 1 | mrppqcllht pslaspllll llwllgggvg aegredaell vtvrggrlrg irlktpggpv |
| 61 | saflgipfae ppmgprrflp pepkqpwsgv vdattfqsvc yqyvdtlypg fegtemwnpn |
| 121 | relsedclyl nvwtpyprpt sptpvlvwiy gggfysgass ldvydgrflv qaertvlvsm |
| 181 | nyrvgafgfl alpgsreapg nvglldqrla lqwvqenvaa fggdptsvtl fgesagaasv |
| 241 | gmhllsppsr glfhravlqs gapngpwatv gmgearrrat qlahlvgcpp ggtggndtel |
| 301 | vaclrtrpaq vlvnhewhvl pqesvfrfsf vpvvdgdfls dtpealinag dfhglqvlvg |
| 361 | vvkdegsyfl vygapgfskd neslisraef lagvrvgvpq vsdlaaeavv lhytdwlhpe |
| 421 | dparlreals dvvgdhnvvc pvaqlagrla aqgarvyayv fehrastlsw plwmgvphgy |
| 481 | eiefifgipl dpsrnytaee kifaqrlmry wanfartgdp neprdpkapq wppytagaqq |
| 541 | yvsldlrple vrrglraqac afwnrflpkl lsatdtldea erqwkaefhr wssymvhwkn |
| 601 | qfdhyskqdr csdl |

The amino acid sequence of T30 (which corresponds to the last 30 amino acid residues of SEQ ID No: 1) is provided herein as SEQ ID No: 2, as follows:—

[SEQ ID No: 2]
KAEFHRWSSYMVHWKNQFDHYSKQDRCSDL

The amino acid sequence of T14 (which corresponds to the 14 amino acid residues located towards the end of SEQ ID No: 1, and lacks the final 15 amino acids found in T30) is provided herein as SEQ ID No: 3, as follows:—

[SEQ ID No: 3]
AEFHRWSSYMVHWK

Preferably, therefore, the peptide of SEQ ID No: 3, or a variant or fragment thereof is T14. Most preferably, the methods and apparatuses of the invention involve detection of soluble peptide comprising or consisting of SEQ ID No: 3.

However, fragments of T14 (SEQ ID No: 3) are also detectable in the methods and apparatuses of the invention, and can act as diagnostic or prognostic markers used in accordance with the invention.

Thus, in one embodiment, a fragment of SEQ ID No: 3 preferably comprises an amino acid sequence of SEQ ID No: 4 (i.e. T7), i.e. SYMVHWK.

In another embodiment, a fragment of SEQ ID No: 3 preferably comprises an amino acid sequence of SEQ ID No: 5 (i.e. T8), i.e. SSYMVHWK.

In another embodiment, a fragment of SEQ ID No: 3 preferably comprises an amino acid sequence of SEQ ID No: 6 (i.e. T9), i.e. WSSYMVHWK.

In another embodiment, a fragment of SEQ ID No: 3 preferably comprises an amino acid sequence of SEQ ID No: 7 (i.e. T10), i.e. RWSSYMVHWK.

In another embodiment, a fragment of SEQ ID No: 3 preferably comprises an amino acid sequence of SEQ ID No: 8 (i.e. T11), i.e. HRWSSYMVHWK.

In another embodiment, a fragment of SEQ ID No: 3 preferably comprises an amino acid sequence of SEQ ID No: 9 (i.e. T12), i.e. FHRWSSYMVHWK.

In another embodiment, a fragment of SEQ ID No: 3 preferably comprises an amino acid sequence of SEQ ID No: 10 (i.e. T13), i.e. EFHRWSSYMVHWK.

In other words, although detection of T14 (i.e. SEQ ID No: 3) is preferred, the invention may also rely on detection of one or more of any of T7-T13 (i.e. SEQ ID No: 4-10).

Preferably, the methods or apparatuses of the invention comprise determining the Braak stage of the subject. The subject may be any of Braak stage I, II or III. Preferably, the subject is Braak stage I. Preferably, the subject is Braak stage II. Preferably, the subject is Braak stage III.

Preferably, the subject is a living subject. Up until now, it is only possible to Braak stage deceased subjects, and so the invention described herein is a significant advance over currently available approaches.

Thus, preferably, the methods comprise:
(a) analysing, in the sample obtained from the test subject, the concentration of the peptide comprising or consisting of SEQ ID No: 3 (T14), or a variant or fragment thereof; and
(b) comparing this concentration with a reference value from a control population of deceased subjects having known Braak stages for concentrations of soluble peptide comprising or consisting of SEQ ID No: 3, or a variant or fragment thereof,
wherein the Braak stage of the living test subject is determined by comparing the concentration of the peptide comprising or consisting of SEQ ID No: 3, or a variant or fragment thereof, with the respective reference value that is associated with a Braak stage.

It will be appreciated that the methods and apparatuses of the invention may be used to determine and monitor disease progression in a method well-correlated with Braak stage. Advantageously, the results described in the Examples supports the inventors' hypothesis that detection of the peptide of SEQ ID No: 3 in individuals can be used to determine the Braak stage of a living subject. Currently, Braak staging can only be performed on a post-mortem brain, and so the methods and apparatuses of the invention provide a significant advance over these current methods. Use of the T14 biomarker (i.e. the peptide of SEQ ID No: 3, or a variant or fragment thereof) allows the determination of the Braak stage of a patient with a very high degree of specificity and sensitivity from a non-invasive, easily repeatable, cost-effective procedure, such as blood, urine or CSF collection, and therefore allows routine screening, diagnosis of Braak stage and appropriate intervention with therapeutic treatment.

It will be appreciated that Braak staging has six stages based on the location of neurofibrillary tangles, with Braak stage o corresponding to a healthy subject. Stages I and II relates to early stage disease and is when neurofibrillary tangles are limited to the transentorhinal region of the brain. Braak stages I and II are pre-symptomatic, and, as shown in FIG. 7, the inventors have surprisingly demonstrated it is possible to detect the T14 in samples at each of these stages. Stages III and IV define neurofibrillary tangle involvement in the limbic regions, which includes the hippocampus, and stages V and VI are when the neurofibrillary tangles are extensive in the neocortical regions of the brain. Accordingly, the methods and apparatus of the invention can be used to determine Braak stage o, I, II, III, IV, V or VI of the living subject. Braak staging is a good method for recording progression of Parkinson's and Alzheimer's diseases in the post mortem brain, and is currently far more reliable than any ante-mortem method.

It will also be appreciated that the methods of the invention are useful for enabling a clinician to precisely diagnose the stage of neurodegeneration and/or cognitive decline, and therefore make informed decisions with regards to the best course of treatment for the patient based on the concentration of T14 detected in the sample or on their Braak stage. In addition, the methods are useful for monitoring the efficacy of a putative treatment for neurodegeneration and cognitive decline. Hence, the apparatuses of the invention are useful for providing a prognosis of the subject's condition, such that the clinician can carry out the treatment according to the third or seventh aspect. The apparatuses may also be used to monitor the efficacy of a putative treatment for neurodegeneration and cognitive decline. The methods and the apparatuses are therefore very useful for guiding a treatment regime for the clinician, and to monitor the efficacy of such a treatment regime.

Preferably, the concentration of: (i) a soluble peptide comprising or consisting of SEQ ID No: 3 (T14), or a variant or fragment thereof and/or (ii) an aggregated peptide comprising or consisting of SEQ ID No: 3, or a variant or fragment thereof, is analysed, and the Braak stage of the living test subject is determined by comparing the concentration of either the soluble or aggregated peptide comprising or consisting of SEQ ID No: 3, or a variant or fragment thereof, with the respective reference value that is associated with a Braak stage.

Preferably, soluble peptide is analysed. In some embodiments, a higher concentration of soluble peptide comprising or consisting of SEQ ID No: 3, or a variant or fragment thereof, compared to the reference value is indicative of a later Braak stage. In other words, the higher the concentration of soluble T14, the greater the correlation with a later Braak stage, such as stage IV, V or VI, of the living subject. Soluble peptide SEQ ID No: 3 (T14), or a variant or fragment thereof, is preferably determined using ELISA, most preferably performed on a saliva, nasal fluid/discharge or pin-prick blood plasma sample taken from the subject.

However, preferably a lower concentration of soluble peptide comprising or consisting of SEQ ID No: 3, or a variant or fragment thereof, compared to the reference value is indicative of a later Braak stage. In other words, the lower the concentration of soluble T14, the greater the correlation with a later Braak stage, such as stage IV, V or VI, of the living subject. Soluble peptide SEQ ID No: 3 (T14), or a variant or fragment thereof, is preferably determined using lateral flow or ELISA, most preferably performed on a saliva, nasal fluid/discharge or pin-prick blood plasma sample taken from the subject.

Thus, preferably, a lower concentration of soluble peptide comprising or consisting of SEQ ID No: 3 or a variant or fragment thereof compared to the reference value is indicative that the patient is beta amyloid positive, and/or a higher concentration of a soluble peptide comprising or consisting of SEQ ID No: 3 or a variant or fragment thereof compared to the reference value is indicative that the patient is beta amyloid negative. Preferably, a lower concentration of a soluble peptide comprising or consisting of SEQ ID No: 3 or a variant or fragment thereof compared to the reference value is indicative that the subject is cognitively impaired, and/or a higher concentration of a soluble peptide comprising or consisting of SEQ ID No: 3 or a variant or fragment thereof compared to the reference value is indicative that the patient is cognitively normal.

Hence, in a preferred embodiment, a lower concentration of soluble peptide comprising or consisting of SEQ ID No: 3, or a variant or fragment thereof, compared to the reference value is indicative of Braak stage IV. Alternatively, in another preferred embodiment, a lower concentration of soluble peptide comprising or consisting of SEQ ID No: 3, or a variant or fragment thereof, compared to the reference value is indicative of Braak stage V. Alternatively, in another preferred embodiment, a lower concentration of soluble peptide comprising or consisting of SEQ ID No: 3, or a variant or fragment thereof, compared to the reference value is indicative of Braak stage VI.

The inventors have also surprisingly discovered that the concentration of T14 differs amongst earlier Braak stages (see FIG. 7), therefore demonstrating that T14 levels can be used to determine the earlier pre-symptomatic Braak stages (e.g. I, II and III) in living patients. Accordingly, in one embodiment, a lower concentration of soluble peptide comprising or consisting of SEQ ID No: 3, or a variant or fragment thereof, compared to the reference value is indicative of Braak stage I. Alternatively, in another embodiment, a lower concentration of soluble peptide comprising or consisting of SEQ ID No: 3, or a variant or fragment thereof, compared to the reference value is indicative of Braak stage II. Alternatively, in another embodiment, a lower concentration of soluble peptide comprising or consisting of SEQ ID No: 3, or a variant or fragment thereof, compared to the reference value is indicative of Braak stage III.

In another embodiment, a higher concentration of a soluble peptide comprising or consisting of SEQ ID No: 3 or a variant or fragment thereof compared to the reference value is indicative that the patient is beta amyloid positive, and/or a lower concentration of a soluble peptide comprising or consisting of SEQ ID No: 3 or a variant or fragment thereof compared to the reference value is indicative that the patient is beta amyloid negative. Preferably, a higher concentration of a soluble peptide comprising or consisting of SEQ ID No: 3 or a variant or fragment thereof compared to the reference value is indicative that the subject is cognitively impaired, and/or a lower concentration of a soluble peptide comprising or consisting of SEQ ID No: 3 or a variant or fragment thereof compared to the reference value is indicative that the patient is cognitively normal.

The skilled technician will appreciate how to measure the concentrations of T14 peptide (either soluble or aggregated) in a statistically significant number of control individuals, and the concentration of T14 in the test subject, and then use these respective figures to determine the Braak stage of the test subject. Comparing the levels of the peptide of SEQ ID No: 3 (i.e. T14) in a sample (preferably, blood plasma) collected from a large group of well-characterised Braak stage o individuals (i.e. no disease or “normal” health) as close as possible to subject death may be a preferred method of defining a control population (i.e. cohort) of the reference value.

In another embodiment, after collection of post-mortem CSF samples from a sufficient number of control and Alzheimer's Disease subjects (e.g. n>50 for each group), their soluble and aggregated T14 levels may be measured by ELISA and Western Blot, respectively. These levels are preferably calibrated to the subject's Braak stages (=o for controls) to determine the relationship between the change in T14 and incremental increase in Braak staging. The resultant standard curve can be used by future ex-vivo CSF samples from living patients to extrapolate their Braak stage from their T14 levels in the patient's asymptomatic stage of disease.

In another embodiment, samples may be required from a sufficient number of control and AD subjects (e.g. n>200 for each group). Their soluble and/or aggregated T14 levels may be measured by ELISA and Western Blot, respectively. These levels may be normalised to that from a healthy subject. This normalisation step may not be needed. Next, the range of control and AD values may be plotted with confidence intervals. Single samples from patients with no disease symptoms may be diagnosed or disease progression predicted by detecting whether or not T14 values falling within or outside of control T14 ranges or falling within or outside AD T14 ranges.

Accordingly, the inventors have realised that the difference in concentrations of T14 between the normal and raised/lowered levels, for aggregated or soluble T14, respectively, can be used as a physiological marker, to determine the Braak stage of a living subject. It will be appreciated that if a subject has a lowered concentration of soluble T14 which is considerably lower than the reference soluble T14 concentration, or a raised concentration of aggregated T14 which considerably higher than the reference aggregated T14 concentration, then this would be indicative of a higher Braak stage. The inventors have also discovered that the concentration of T14 differs amongst earlier Braak stages, therefore demonstrating that T14 levels can be used to determine the earlier pre-symptomatic Braak stages (I, II and III) in living patients.

By way of example, the decrease in concentration of soluble T14 from the reference concentration may be at least 10%, preferably at least a 20% decrease, more preferably at least a 30% decrease, even more preferably at least a 40% decrease, and most preferably a decrease of at least 50% from the reference value concentration. Such decreases in soluble T14 concentrations infer that the test subject will have a higher Braak stage. Alternatively, the increase in concentration of aggregated T14 from the reference concentration may be approximately at least 10%, preferably about at least a 20% increase, more preferably at least a 30% increase, even more preferably at least a 40% increase, and most preferably an increase of at least 50% from the reference value concentration. Such increases in aggregated T14 concentrations infer that the test subject will have a higher Braak stage. Accordingly, a clinician would be able to make an informed decision as to the preferred course of treatment required, for example, the type and dosage of the therapeutic agent according to the third or seventh aspect to be administered.

In another embodiment, it will be appreciated that if a subject has a raised concentration of soluble T14 which is considerably higher than the reference soluble T14 concentration, or a lower concentration of aggregated T14 which considerably lower than the reference aggregated T14 concentration, then this would be indicative of a lower Braak stage.

By way of example, the increase in concentration of soluble T14 from the reference concentration may be at least 10%, preferably at least a 20% increase, more preferably at least a 30% increase, even more preferably at least a 40% increase, and most preferably a increase of at least 50% from the reference value concentration. Such increase in soluble T14 concentrations infer that the test subject will have a higher Braak stage. Alternatively, the decrease in concentration of aggregated T14 from the reference concentration may be approximately at least 10%, preferably about at least a 20% decrease, more preferably at least a 30% decrease, even more preferably at least a 40% decrease, and most preferably an decrease of at least 50% from the reference value concentration. Such decreases in aggregated T14 concentrations infer that the test subject will have a higher Braak stage. Accordingly, a clinician would be able to make an informed decision as to the preferred course of treatment required, for example, the type and dosage of the therapeutic agent according to the third or seventh aspect to be administered.

The methods or apparatuses of the invention may further comprise measuring the rate of cognitive decline by a Mini Mental State Examination (MMSE) score and/or a Preclinical Alzheimer Cognitive Composite (PACC) score.

MMSE is a questionnaire that is administered virtually universally in those with suspected AD, and in broader study cohorts, as a measure of cognitive impairment. It is widely considered a gold standard for diagnosis in AD due to its ease of application with little training required, repeatability, validity and reliability. It is also particularly useful when considering the longitudinal assessment of AD and its progression. Repeated measures of MMSE score, collected at regular intervals, may be used to calculate a rate of cognitive decline for the subject. Then, a linear regression is preferably performed to calculate the slope of MMSE score change over time, and it is this slope which is interpreted as the rate of cognitive change (decline/incline). Preferably, therefore, cognitive decline is measured in terms of MMSE score. The slope may be calculated in points dropped on the MMSE score per month.

The PACC test, on the other hand, combines tests that assess episodic memory, timed executive function, and global cognition. It is the primary outcome measure for the first clinical trial in preclinical AD.

The methods of the invention may further comprise a step of age-adjusting the T14 concentrations from the test subject, be they soluble T14 or aggregated T14, against the corresponding reference value.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with any of the sequences described herein.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, 2, 3, 4, but less than 5, 10, 20, 50 or 100 amino acids from the sequence shown in, for example, SEQ ID No: 3.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 shows (from left to right) a nasal swab to obtain nasal discharge or fluid, a mouth swab to obtain oral fluid, such as saliva, and a device for making a pin-prick to obtain a pin-prick blood sample. Each of these sample types are known as peripheral tissues.

FIG. 2 shows T14 immunoassay data obtained from saliva samples from six humans (labelled 1-6) using the mouth swab shown in FIG. 1.

FIG. 3 shows a perspective view of a lateral flow test cartridge with a sample dropper for delivering sample (e.g. pin prick blood, nasal fluid or saliva shown in FIG. 2) to a sample aperture(S).

FIG. 4 shows a side view of a first embodiment of a lateral flow apparatus of the invention referred to as an inhibition LFT (wet format) for detecting the T14 peptide biomarker.

FIG. 5 shows a side view of a second embodiment of the lateral flow apparatus of the invention referred to as a sandwich LFT (wet format) for detecting the T14 peptide biomarker.

FIG. 6 shows perspective views of embodiments of a desk top reader (on the left) or a smartphone running an app (on the right) for reading the LFT cartridge shown in FIG. 3 following either the inhibition LFT of FIG. 4 or the sandwich LFT of FIG. 5. These can be used to quantify the amount of T14 in the sample.

FIG. 7 shows three illustrations of the brain with clinical symptoms and corresponding Braak stage (from left to right), as well as corresponding Western blots for hippocampal T14 peptide at Braak stage I, II and VI, showing an increase at the later stages.

FIG. 8 shows the theranostic interplay between detecting T14 levels using the method of the invention (e.g. LFT) and therapeutic intervention by an administered acetylcholinesterase inhibitor, or N-methyl-D-aspartate (NMDA) antagonist, or a cyclic peptide (known as NBP-14 cyclic peptide, as described in WO2015/004430) in order to achieve a permanent prevention of symptom onset.

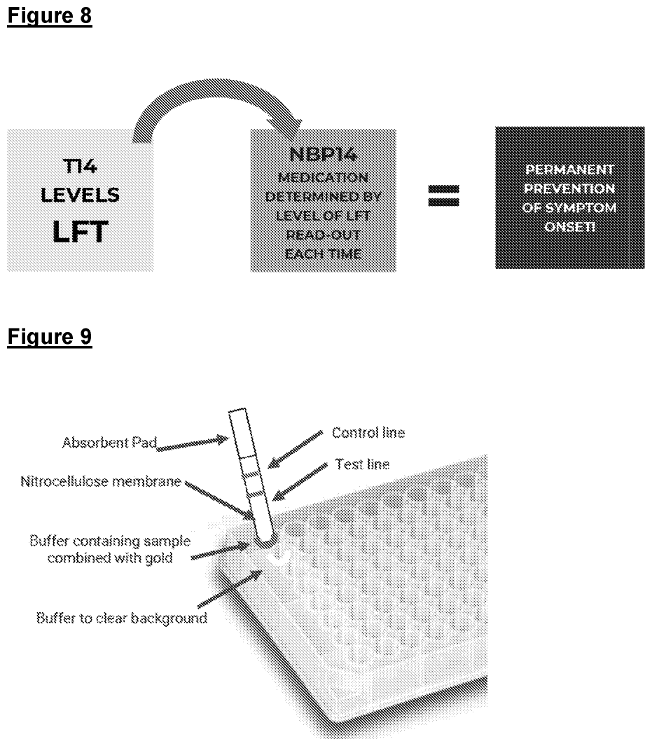

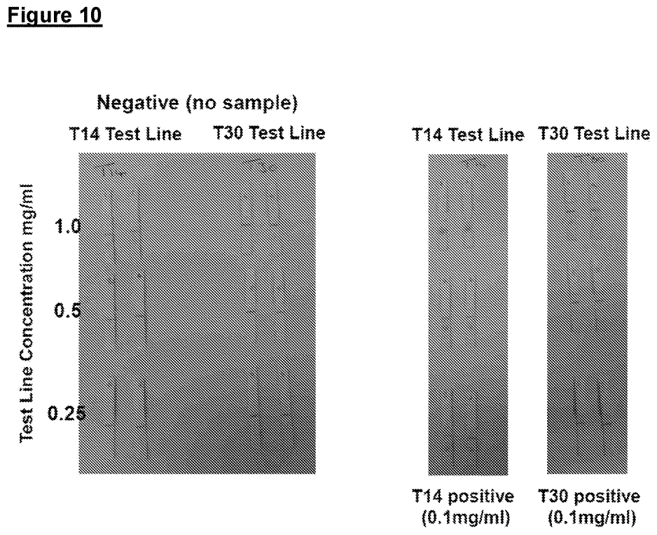

FIG. 9 shows the apparatus used to carry out a wet reagent or “half dipstick” test method.

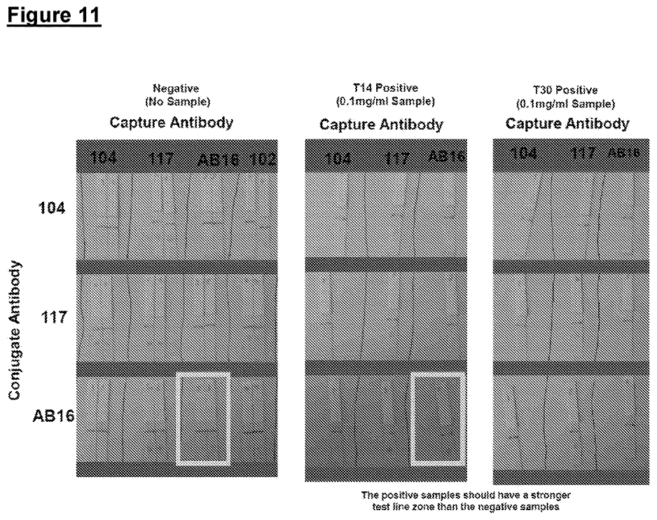

FIG. 10 shows the results of inhibition lateral flow assay.

FIG. 11 shows the results of sandwich lateral flow assay.

### EXAMPLES

The inventors have observed that a T14 biomarker peptide is surprisingly present in very small, but detectable, concentrations in saliva, nasal discharge and pin-prick blood. Such sample types may be commonly analysed using a lateral flow test (LFT), which, unlike CSF and venous/arterial blood, are painless, socially acceptable by the general public, low cost, can be used frequently, and provide very fast and accurate results.

### Materials and Methods

### Generation of the Braak Stage Data, CSF Data & Saliva Data

### Western Blot—Brain Tissue, CSF & Saliva

The human hippocampus samples and the Post-mortem CSF analysed were provided by the Oxford Brain Bank, and classified as Braak stages o, I, II, III, V, or VI. Saliva samples were collected from 6 donors and placed in separate tubes.

### Brain Tissue:

Western blots were carried out as described here: Briefly, ~0.2 g of frozen human brain tissue was thawed and homogenized in ice-cold Neuronal Protein Extraction Reagent supplemented with phosphatase and protease inhibitors. Buffer, 1 mL, was added per 0.2 g of brain tissue. Homogenate was centrifuged (16,000×g, 30 minutes, 4° C.) and supernatant quantified for protein.

The Thermo Scientific Pierce 660 nm Protein Assay was used to determine the protein. This assay is a ready-to use, detergent- and reducing agent-compatible assay to quickly measure total protein concentration compared to a protein standard of bovine serum albumin. For the assay, 10 ml of each human brain homogenate sample were added to a microtiter 96 well-plate followed by the addition of 150 ml of Pierce assay. After 5 min incubation, the absorbance was measured at 660 nm in a Vmax plate reader (Molecular Devices, Wokingham, UK) and the results of optical density were extrapolated to the standard curve of BSA to obtain mg per ml.

One hundred micrograms of protein were mixed with 4×Laemmli sample buffer (62.5 mM Tris-HCl pH 6.8, 10% glycerol, 1% LDS, 0.005% Bromophenol Blue, 50 mM dithiothreitol [DTT]), heated to 50° C. (10 minutes), and loaded onto 4%-20% Mini-PROTEANTGX Precast Protein Gels, 10-well, 50 μL. Proteins were separated by electrophoresis, transferred to PVDF (0.45 μm) membrane, and blocked (1 hour; RT) with 5% Blotting Grade Blocker Non-Fat Milk in Tris-buffered saline plus 0.05% Tween20 (TBS-T0.05%). The membrane was incubated overnight with T14 antibody (stock 1 mg/mL, dilution 1:1000) as described previously in WO 2016/156803. Membranes were washed and incubated with secondary antibody (1:10000). After washing, immunoreactive protein was visualised using an enhanced chemoluminescence-based detection kit, following the manufacturer's protocol (Thermo Scientific Pierce ECL Plus Western Blotting Substrate) and a CCD Camera (G-Box, Syngene, Cambridge, UK) gel system. Scanned blots were analysed using GensSnap software (Syngene, Cambridge, UK) and dot densities were expressed as a percentage of those taken from the control. Bands were quantified using ImageJ, and unpaired tests were performed using GraphPad Prism9.0.

### Detecting T14 in CSF:

CSF samples were used directly for electrophoresis. Protein was determined as above using the Thermo Scientific Pierce 660 nm Protein Assay. For each sample, 20 μg of CSF samples was mixed with sample buffer (0.5 M Tris HCl, pH 6.8, 10% glycerol, 2% (w/v) sodium dodecyl sulphate, 5% (v/v) 2-b-mercaptoethanol, 0.05% bromophenol blue, final concentrations), boiled for 10 min, and loaded onto a 10% acrylamide gel. Proteins were separated by electrophoresis until the elution of the migration front to allow proper separation of high molecular weight fragments. Proteins were then transferred from gels to polyvinylidene fluoride sheets (ThermoFisher). These sheets were blocked for 1 h at room temperature with 5% defatted milk in Tris-buffered saline buffer plus 0.05% Tween 20 (TBS-T buffer). They were then incubated overnight with T14 antibody (1:1000, Genosphere), diluted in TBS-T buffer plus 5% defatted milk. Thereafter, the membranes were washed with TBS-T buffer and incubated for 45 min with anti-rabbit IgG Horseradish Peroxidase (HRP) conjugated secondary antibody (ab6721 Abcam, Cambridge, UK 1:5000 dilution). After washing, immunoreactive protein was visualised using an enhanced chemoluminescence-based detection kit, following the manufacturer's protocol (Thermo Scientific Pierce ECL Plus Western Blotting Substrate) and a CCD Camera (G-Box, Syngene, Cambridge, UK) gel system. Scanned blots were analysed using GensSnap software (Syngene, Cambridge, UK) and dot densities were expressed as a percentage of those taken from the control. Bands were quantified using ImageJ, and unpaired tests were performed using GraphPad Prism9.0.

### Detecting T14 in Saliva:

Saliva samples were obtained from 6 donors by using a mouth swab or spitting directly into a tube. For the Western blots, 10 ul total protein (unknown concentration) was loaded with 5 ul of sample buffer (0.5 M Tris HCl, pH 6.8, 10% glycerol, 2% (w/v) sodium dodecyl sulphate, 5% (v/v) 2-b-mercaptoethanol, 0.05% bromophenol blue, final concentrations). Then the sample was boiled for 10 min, and loaded onto a 4%-20% Mini-PROTEANTGX Precast Protein Gels, 10-well, 50 μL. Proteins were separated by electrophoresis until the elution of the migration front to allow proper separation of high molecular weight fragments. Proteins were then transferred from gels to polyvinylidene fluoride sheets (ThermoFisher). These sheets were blocked for 1 h at room temperature with 5% defatted milk in Tris-buffered saline buffer plus 0.05% Tween 20 (TBS-T buffer). They were then incubated overnight with T14 antibody (1:1000, Genosphere), diluted in TBS-T buffer plus 5% defatted milk. Thereafter, the membranes were washed with TBS-T buffer and incubated for 45 min with anti-rabbit IgG Horseradish Peroxidase (HRP) conjugated secondary antibody (ab6721 Abcam, Cambridge, UK 1:10,000 dilution). After washing, immunoreactive protein was visualised using an enhanced chemoluminescence-based detection kit, following the manufacturer's protocol (Thermo Scientific Pierce ECL Plus Western Blotting Substrate) and a CCD Camera (G-Box, Syngene, Cambridge, UK) gel system. Scanned blots were analysed using GensSnap software (Syngene, Cambridge, UK) and dot densities were expressed as a percentage of those taken from the control. Bands were quantified using ImageJ, and unpaired tests were performed using GraphPad Prism9.0. For the immunoneutralised experiment, T14 primary antibody (1:1000) was incubated with 1 mg/ml T14 peptide for 3 h at room temp on rocker before adding to the membrane.

### Detecting T14 in Nasal Discharge:

The inventors believe that the concentration of T14 in nasal discharge (or saliva) is about 20-40 ng of T14/mg protein. For the LFT embodiment, there are several embodiments of the device based on using antibody detection systems. The technology has been developed with a polyclonal and a monoclonal IgG antibody, and three different types of immunoassay, i.e. Indirect, Competitive and Sandwich. The antibodies have been shown to work well in Western Blot, ELISA, AlphaLISA, immunohistochemical staining to detect both synthetic and endogenous T14, and the binding specificity and epitope has been determined as a good candidate for Indirect and competitive immunoassays.

### Lateral Flow Test (LFT) Reagents

(Ab16) Affinity purified polyclonal anti-peptide T14 antibodies;
Monoclonal liquid THK-1-102 purified rabbit IgG (~15 μl);
Monoclonal liquid THK-1-104 purified rabbit IgG (~600 μl);
Monoclonal liquid THK-1-117 purified rabbit IgG (1 ml);
T14 peptide (powder form/lyophilised trifluoroacetate salt (1 mg); and
T30 peptide (powder form/lyophilised trifluoroacetate salt (1 mg).

### Conjugation of Antibodies for LFT

The detector reagent was a 40 nm gold particle which is passively conjugated to the antibodies. Conjugates were prepared for: AB16 (rabbit polyclonal), AB117 (rabbit monoclonal) and AB104 (rabbit monoclonal).

A titration of pH and antibody loading onto 40 nm gold colloid was completed.

Preferred conditions for the conjugates were:

| | Antibody | Conjugation | Concentration |
|---|---|---|---|
| Antibody | loading | buffer | (OD) |
| AB16 (polyclonal) | 18 μg | PBS | 2.08 |
| AB117 (monoclonal) | 14 μg | Carbonate | 1.28 |
| AB104 (monoclonal) | 18 μg | carbonate | 1.66 |

Each of the antibodies was conjugated to 40 nm gold colloid.

### LFT Method

A wet reagent or “half dipstick” test method was used, as shown in FIG. 9. In this format, a gold conjugate is in liquid form (in the well) rather than dry enabling high throughput of the test conditions. The test line is “dotted” onto the membrane by pipette to facilitate evaluation of a range of conditions.

The nitrocellulose membrane was CN180.

### Test Lines:

A) Inhibition Assay: T14 peptide
T30 peptide
B) Sandwich Assay: Rabbit polyclonal, AB16
Rabbit monoclonal, AB117
Rabbit monoclonal, AB104
Rabbit monoclonal, AB102

### Control Line: Goat Anti-Rabbit Antibody

### Inhibition LFT Method

### Membrane Preparation:

Peptide (T14 or T30) was diluted to 1, 0.5 and 0.25 mg/ml in deionised water. The peptide was “dotted” onto CN180 nitrocellulose membrane (1 μl at each concentration) and the membrane dried for 15 minutes at 37° C. The goat anti-rabbit antibody was applied to the membrane as a control line.

### Test Method:

(i) 20 μl gold conjugate (neat) and 20 μl TBST (Tris buffered saline 1% Tween 20) added to Well 1;
(ii) The dipstick was added to Well 1;
(iii) The dipstick was run to completion (no liquid remaining); and
(iv) The dipstick was added to Well 2 containing 20 μl TBST only to wash the test strip.

### Sandwich LFT Method

### Membrane Preparation:

Each antibody was “dotted” onto CN180 membrane (1 μl neat, i.e. 1 mg/ml, concentration) and the membrane dried for 15 mins at 37° C. The goat anti-rabbit antibody was applied to the membrane as a control line. Conjugate and test line antibody combinations were then evaluated in a matrix study in the lateral flow format.

### Test Method (Sequential Sample Addition):

(i) 10 μl peptide (either T14 or T30 at 0.1 mg/ml) and 10 μl TBST (Tris buffered saline 1% Tween 20) added to Well 1;
(ii) The dipstick was added to Well 1;
(iii) The dipstick was run to completion (no liquid remaining);
(iv) The dipstick was added to Well 2 containing 20 μl conjugate;
(v) The dipstick was run to completion (no liquid remaining); and
(vi) The dipstick was added to Well 3 containing 20 μl TBST.

### AlphaLISA Detection of T14-Alpha-7 Complex

Samples were extracted from homogenized human brain tissue using PerkinElmer lysis buffer (AL003C) and the protein concentration determined using the BCA method. For 100 mg tissue homogenization, 1 mL of lysis buffer was used. Five cycles of 40 second pulse, and 10 second breaks, on a shielded homogenizer were used for each sample. Samples were centrifuged at 40° C., 15000 rpm (15 minutes) for supernatants; these were diluted in PerkinElmer Assay buffer (ALoooF) and used to measure T14-alpha-7 nicotinic receptor complexes in the presence of NBP14 (concentrations 0.065 μM-900 μM) (Genosphere) with AlphaLISA following the manufacturer's protocol. The antibodies were biotinylated BTX (B1196; Invitrogen Life Technologies, Waltham, MA, USA) on SA-donor beads and anti-rabbit T14 (Genosphere) on acceptor beads; results read in an AlphaLISA Reader (model #EnSpire 2300 Multilabel Reader; PerkinElmer).

### Example 1—Lateral Flow Test (LFT) Implementation to Detect T14 Peptide

Referring to FIG. 1, there are shown three different peripheral tissue sample types which can be readily used for subsequent lateral flow tests, and these include a nasal swab (2) to obtain nasal discharge or nasal fluid from the nasal passage, an oral swab (4) for obtaining oral fluid, such as saliva from the buccal cavity or mouth, or a finger stick (6) for creating a small prick in the skin to obtain blood (capillary or whole blood), usually from a finger (but it could be elsewhere on the body).

By way of example, direct spitting or a mouth swab (2) was used to obtain oral fluid from six test subjects, which was then analysed. As shown in FIG. 2, the inventors have surprisingly demonstrated that the T14 biomarker peptide (SEQ ID No: 3) is surprisingly present in very small, but detectable, concentrations in the saliva. The western blot results show the detection of a T14 band in all the samples. To verify that the band is T14 and not cross contamination of the antibody with another molecule, the samples were immunoneutralised (as described in the method above) to show that the antibody is specifically detecting T14. The concentration of the T14 peptide in the saliva was approximately 20-40 ng T14 peptide per mg protein in the sample. Accordingly, the inventors set out to analyse T14 in the sample using a lateral flow test (LFT), and believe that pin prick blood and nasal discharge could also be used as a sample instead of saliva.

Therefore, once the sample (nasal discharge, blood or saliva) has been obtained from the subject, it is then inserted into a sample tube 8 to which a buffering solution (sample buffer: 0.5 M Tris HCl, pH 6.8, 10% glycerol, 2% (w/v) sodium dodecyl sulphate, 5% (v/v) 2-b-mercaptoethanol, 0.05% bromophenol blue, final concentrations) is than added and mixed for sufficient time so that the sample, and any T14 peptide therein, is suitably suspended and dispersed. Referring to FIG. 3, there is shown an LFT cartridge (10) which has, towards one end, a sample aperture (12, S) into which a few drops of buffered sample solution is administered from the tube (8). The cartridge (10) has a window (14) through which a test line (16, T) and control line (18, C) can be viewed. The mechanism of lateral flow detection of T14 peptide will now be described below.

FIGS. 4 and 5 show two different embodiments of lateral flow apparatus, namely an inhibition lateral flow test (20) shown in FIG. 4, and a sandwich lateral flow test (22) shown in FIG. 5. LFT technology described below, using either a polyclonal or monoclonal antibody, is qualitative, semi-quantitative or quantitative.

### Inhibition Lateral Flow Test (LFT)

Referring to FIG. 4, in the inhibition LFT (20), the LFT cartridge (10) has a sample nitrocellulose membrane CN180 pad (24) onto which sample (26) is placed through the aperture (12). The sample (26) may or may not comprise sample T14 peptide (15), and therefore creates different results as discussed below. Disposed adjacent to the sample pad (24) there is provided a conjugate pad (28) on which anti-T14 antibodies (30) are disposed (labelled with a 40 nm gold nanoparticle colloid passively conjugated to the T14 immuno-specific antibody). For example, the anti-T14 antibody (30) can be as described in WO 2016/156803.

The cartridge (10) has an LFT fixed T14 peptide test line (16) consisting of pre-fixed T14 peptide (17) attached thereto. Laterally spaced apart from the fixed T14 peptide test line (16) there is provided a control line (18) consisting of a secondary anti-species labelled antibody (31), which is not specific for T14 (i.e. goat anti-chicken IgY polyclonal antibody). An upper wick (32) is provided towards the opposite end of the cartridge (10), and functions to draw the sample (26) solution laterally across the cartridge (10).

As illustrated in the upper part of FIG. 4 (showing a positive result for T14 detection), when the sample (26) includes sample T14 peptide (15), and it flows laterally over the conjugate pad harbouring the anti-T14 antibodies (30), it is captured by the anti-T14 antibody (30) creating a conjugate of T14-antibody (34). This T14-antibody conjugate (34) is unable to bind to the fixed T14 peptide (17) that is affixed to the test line (16), whereas anti-T14 antibody (30) that is not bound to sample T14 peptide (15) in the sample (26) can flow and bind to the anti-species control (31) affixed to the control line (18). The sample T14 (15) is therefore arresting the antibody (30) which cannot bind the fixed T14 (17) attached to the cartridge (10). Accordingly, a single line develops on the cartridge (10) as a positive result, as shown to the upper right of FIG. 4.

As illustrated in the lower part of FIG. 4 (showing a negative result for T14 detection), when the sample (26) does not include sample T14 peptide (15), the sample (26) flows laterally over the conjugate pad harbouring the anti-T14 antibodies (30). As there is no sample T14 (15) present to be captured by the anti-T14 antibody (30), no conjugate of T14-antibody (34) is created, as in the positive results discussed above. Accordingly, all unbound anti-T14 antibody (30) can flow and bind to the LFT fixed T14 peptide (17) that is affixed to the test line (16) as well as to the anti-species control (31) affixed to the control line (18). Accordingly, two spaced apart lines develop on the cartridge (10) as a negative result, as shown to the lower right of FIG. 4.

Referring now to FIG. 10, there are shown the results of the “half dipstick” LFT method represented in FIG. 9. As can be seen in FIG. 10, the antigen was able to bind to the membrane in the inhibition format. As can be seen, LFT was able to detect both T14 and T30 peptides. A reduction in signal intensity in the test line zone was seen when the amount of peptide on the membrane was reduced from 1 mg/ml to 0.25 mg/ml which illustrates specific binding. Some non-specific binding, however, was observed at times.

### Sandwich Lateral Flow Test (LFT)

Referring now to FIG. 5, the inventors have also developed a sandwich (i.e. two antibody) assay format (22) due to the size of the T14 peptide in which a first antibody (30) binds to a first epitope (36) disposed in one region of the T14 peptide (e.g. at the N-terminus), and a second antibody (38) binds to a second epitope (40) which is spaced apart from the first epitope (e.g. at the C-terminus).

In the sandwich LFT (22), the LFT cartridge (10) has a sample pad (24) onto which sample (26) is placed through the aperture (12). The sample (26) may or may not comprise sample T14 peptide (15). Disposed adjacent to the sample pad (24) there is provided a conjugate pad (28) on which the first anti-T14 antibodies (30) are disposed (labelled with a 40 nm gold nanoparticle colloid passively conjugated to antibody). For example, the first T14 antibody (30) can be as described in WO 2016/156803. These anti-T14 antibodies (30) bind to the first epitope (36) on the T14 peptide, such as the C-terminal residues of T14.

The cartridge (10) has a T14 peptide test line (16) consisting of a pre-fixed, second anti-T14 antibody (38) attached thereto. The second T14 antibody (38) binds to the second epitope (40) on the T14 peptide. For example, the second T14 antibody can bind to the N-terminus of T14.

Laterally spaced apart from the test line (16), the cartridge (10) has a control line (18) consisting of an anti-species labelled antibody (31), which is not specific for T14 (e.g. goat anti-chicken IgY polyclonal antibody), and an upper wick (32) provided towards the opposite end of the cartridge (10), which functions to draw the sample (26) solution laterally across the cartridge (10).

As illustrated in the upper part of FIG. 5 (showing a positive result for T14 detection), when the sample (26) includes T14 peptide (15), and it flows laterally over the conjugate pad (28) harbouring the first anti-T14 antibodies (30), it is captured by the anti-T14 antibody (30) creating a conjugate of T14-antibody (34). This T14-antibody conjugate (34) is itself captured by the second anti-T14 antibody (38) which is secured to the cartridge (10) along the test line (16), because the second epitope (40) is still exposed. Accordingly, a first line develops on the cartridge (10) signifying antibody binding at the T14 test line (16). Furthermore, additional anti-T14 antibody (30) that has not bound to T14 peptide in the sample (26) flows and binds to the anti-species control (31) affixed to the control line (18). Unlike in the inhibition LFT assay (20) described in FIG. 4, in the sandwich assay (22) shown in FIG. 5, because the cartridge (10) does not include any LFT fixed T14 (17), the assay using two antibodies (30, 38) is a direct detection of the sample T14 (15) present in the sample. Accordingly, a second line develops on the cartridge (10) as a positive result, as shown to the upper right of FIG. 5.

As illustrated in the lower part of FIG. 5 (showing a negative result for T14 detection), when the sample (26) does not include T14 peptide (15), and it flows laterally over the conjugate pad harbouring the anti-T14 antibodies (30), there is no T14 (15) present to be captured by the anti-T14 antibody (30), and so no conjugate of T14-antibody (34) is created, as in the positive results discussed above. Accordingly, all unbound anti-T14 antibody (30) flows over and cannot bind to the second T14 antibody (38) affixed to the cartridge (10). The T14 antibody (30) flows to and binds the anti-species control (31) affixed to the control line (18), and creates one line on the cartridge (10) as a negative result, as shown to the lower right of FIG. 5.

Referring now to FIG. 11, there are shown the results of the “half dipstick” LFT method represented in FIG. 9. As can be seen, LFT was able to detect both T14 and T30 peptides. Some non-specific binding was observed in the dipsticks indicating that the test line and conjugate antibodies are interacting. However, these interactions can happen for a number of reasons including sub-optimal conjugation conditions.

The inventors note that there not a significant increase in the test line signal when adding either the T14 or T30 peptide at 0.1 mg/ml. The only exception to this being the AB16 capture conjugate pair which indicated a slight increase with a positive sample (highlighted in the gold boxes).

Qualitative and quantitative analysis of LFT results It will be appreciated from the foregoing that the lateral flow tests described above provide a fast and convenient means for detecting the presence or absence of the T14 peptide (15) in the peripheral bodily sample taken from the subject. The presence of the control line (18) is required to ensure that the test is robust and valid, and the presence or absence of the test line (16) informs the subject of the presence or absence of T14 peptide (15) in their sample. Visually assessing the presence or absence of the test line (16) provides a useful qualitative diagnostic or prognostic test, and visually determining the relative thickness (or darkness in colour) of the test line (16) provides a useful semi-quantitative measure. However, achieving accurate and fully quantitative assessments from merely looking at an LFT result is challenging.

Referring to FIG. 6, there is shown an embodiment of a desk top reader (42, on the left) and an embodiment of a smartphone running an app (44, on the right) for quantitatively measuring the results of the LFT cartridge (10) following the use of either the inhibition LFT of FIG. 4 or the sandwich LFT of FIG. 5. The cartridge may be connected to the reader (42), which runs software for detecting the test line (16) and control line (18) and then, using these concentrations/values, accurately calculating the concentration of T14 peptide (15) in the sample (26). Similarly, the cartridge (10) can be connected to a computer, tablet or smartphone (44) running software for detecting the test line (16) and control line (18) and then calculating the concentration of T14 peptide (15) in the sample (26). Alternatively, a user can take a photograph or the LFT cartridge (10) and the software determines the levels of T14 peptide (15) in the sample (26) based on the strength or intensity of signal in the test line (16) and control line (18).

### Example 2—Braak Staging Using LFT

As shown in FIG. 7, the inventors have surprisingly shown that there is a clear and surprising correlation between the concentration of T14 peptide and the Braak stage of a subject, which, in turn, corresponds to the subject's clinical symptoms. For example, the inventors have now detected T14 in samples obtained from living patients who are at Braak stage I and II (brain pathology) and are asymptomatic (clinical). Braak stages III and IV are patients showing mild symptoms, while Braak stages V and VI correspond to dementia and cognitive decline.

As can be seen in FIG. 7, Western Blots of the Alzheimer's disease hippocampus showed a single T14-reactive band that increased approximately 2-fold from early (Braak o-II) to late stages (Braak V-VI). As can be seen, there is a very clear signal in the Western blot of the Braak stage VI sample. Surprisingly, the data also shows that changes in T14 levels can be used to determine the early Braak stages, I and II. Importantly, therefore, this demonstrates that the concentration of T14 can be used to determine the Braak stage for living asymptomatic patients (i.e. those in Braak stage I and II).

Accordingly, the inventors believe that the lateral flow testing described in Example 1 can be conveniently used to detect T14 peptide, and, based on the concentration of T14, determine the Braak stage of the subject from Braak stage o to Braak stage VI. Given that Braak staging is currently carried out post-mortem, the ability to provide a Braak stage score on a living patient from an LFT test would be hugely beneficial, as discussed in more detail below.

### Example 3—Diagnostic, Prognostic and Therapeutic Uses of LFT Testing

The inventors were surprised that detectable levels of the T14 peptide (SEQ ID No: 3) would be in any peripheral tissues, such as saliva, nasal discharge or pin prick blood, which are suitable for lateral flow testing. As such, there are at least four scenarios in which the methods and apparatuses could be used to test the public:—

1) Prognostic Test-Subjects Already Diagnosed with Alzheimer's Disease

For people who are already confirmed as suffering from neurodegenerative disorder and/or cognitive decline, there is a huge value in the rapid and accurate tests of the invention, which could be used to actively monitor the aetiology or progression of their condition (i.e. prognosis), and hopefully follow improvement, slowing down or even reversal of cognitive decline. Such individuals may be Braak stage III, IV, V or VI.

Referring to FIG. 8, there is shown the inter-relationship between detecting T14 levels using diagnostic or prognostic methods of the invention (e.g. LFT tests, or saliva/pin prick blood testing) and therapeutic intervention with an administered drug for treating or preventing onset of neurodegenerative disorder or cognitive decline. For example, an acetylcholinesterase inhibitor, or N-methyl-D-aspartate (NMDA) antagonist, or a cyclic peptide (known as NBP-14 cyclic peptide, as described in as described in WO2015/004430) could be administered in order to achieve a permanent prevention of symptom onset or delay or even reverse symptoms. Such a test could then be used to influence a more accurate dosage of the therapeutic drug being administered to the patient, and also result in a better patient outcome. This is essentially a companion diagnostic for clinical trials and treatment of patients as a very sensitive monitoring system for efficacy of treatment.

The inventors envisage a quantitative read-out of the levels of T14 that would inform the amount of treatment that needs to be received because a patient could daily, weekly or monthly tests at a frequency that is consistent with the dosage regimen of the therapeutic, and constantly and regularly monitor its effects. One would hope that a subject would see a reduction in the T14 levels as a direct results of the therapeutic being administered, in such a way that a successful outcome would be achieved, either by delaying further symptom onset, preventing symptom onset, or even reversing symptoms, such as cognitive decline.

### 2) Diagnostic Test-Subjects Suspected of Having Alzheimer's Disease

This relates to an accurate and fast diagnostic test for use on people who are suspected (but not yet diagnosed) of having neurodegenerative disorders, i.e. they may be pre-symptomatic (Braak stage I or II). Such a test would be compared with the cost, frequency and efficacy of the cognitive testing methods which are currently deployed, e.g. after patient referral to a memory clinic.

### 3) Asymptomatic Test-Subjects not Suspected of Having Alzheimer's Disease

It would be useful to have a convenient test available for people who are not currently suspected of having neurodegenerative disorder, i.e. they are young and/or pre-symptomatic (Braak stage I or II). The opportunity to have a pre-symptomatic test that could be conducted in a GP surgery, or by a nurse, or even at home, to diagnose neurodegenerative disorders. For example, the LFT test could be routinely conducted (or suggested or requested to be done by employers or the NHS etc.) in a certain age population, or above a certain age, similar to breast cancer or bowel cancer screening. Then, depending on the results, one may be referred to a doctor, specialist medical practitioner.

### 4) Drug Development and Clinical Trials

Pharmaceutical companies would be able save time and costs in their drug development if an accurate measure of disease progression were readily available, in terms of timescales, smaller sample groups, and other value metrics, because the patient would effectively serve as their own control, as it could be monitored how much they had or had not deteriorated since an earlier test (e.g. a day, week or month previously).

### CONCLUSIONS

The inventors have shown that T14 levels are elevated in the brain at the pre-symptomatic stage of Alzheimer's disease, and so have translated this profile for detection in peripheral tissue, such as saliva/nasal fluid, as a basis for a reliable diagnostic for early stage Alzheimer's disease. Accordingly, lateral flow could be used as a quick and convenient means for detecting T14, for diagnosis.

LFT modalities may be either inhibition LFT or sandwich LFT, as demonstrated in FIGS. 4, 5, 9, 10 and 11. The antibody was successfully conjugated to 40 nm gold colloid. The antigen was able to bind to the membrane in the inhibition format and sandwich format. A reduction in signal intensity in the test line zone was seen when the amount of peptide on the membrane was reduced illustrating specific binding.

As such, this first-in-class pre-symptomatic test can serve not only as a diagnostic monitor, but also as a prognostic test. Moreover, the inventors believe that the methods and apparatuses described herein can be used to diagnose a subject before onset of any symptoms of dementia (i.e. in the pre-symptomatic stage), and determine when in the future symptoms may otherwise present. Upon detection of T14 in the pre-symptomatic stages, the subject could immediately administer therapeutic intervention (e.g. a drug or life-style change) and then use the methods and apparatuses as a prognostic measure to monitor and evaluate drug efficacy.

1. A lateral flow method of diagnosing or prognosing a neurodegenerative disorder in a subject, the method comprising using lateral flow to detect, in a sample obtained from a test subject, a peptide comprising or consisting of SEQ ID No: 3 (T14), or a variant or fragment thereof, wherein detecting the presence of the peptide comprising or consisting of SEQ ID No: 3 (T14), or a variant or fragment thereof in the sample indicates that the test subject has a neurodegenerative disorder, or a pre-disposition thereto or a negative prognosis thereof.

2. A lateral flow apparatus, for diagnosing or prognosing neurodegenerative disorder in a subject, the apparatus comprising a lateral flow support for detecting, in a sample obtained from a test subject, a peptide comprising or consisting of SEQ ID No: 3 (T14), or a variant or fragment thereof, wherein detection of the peptide comprising or consisting of SEQ ID No: 3 (T14), or a variant or fragment thereof corresponds to the subject having a neurodegenerative disorder, or a pre-disposition thereto or a negative prognosis thereof.

3. A method or apparatus according to either claim 1 or claim 2, comprising prognosing the progression of the neurodegenerative disorder.

4. A method or apparatus according to any preceding claim, comprising diagnosing a neurodegenerative disorder.

5. A method or apparatus according to any preceding claim, comprising diagnosing pre-symptomatic conditions in the test subject.

6. A method or apparatus according to any preceding claim, comprising conducting a clinical trial to monitor the activity or efficacy of a drug administered to the test subject, and preferably determine how much the subject has, or has not, deteriorated since an earlier test.

7. A method or apparatus according to any preceding claim, wherein the peptide comprising or consisting of SEQ ID No: 3 (T14), or a variant or fragment thereof, is not determined using ELISA.

8. A method or apparatus according to any preceding claim, comprising determining the concentration of soluble peptide comprising or consisting of SEQ ID No: 3, or a variant or fragment thereof.

9. A method or apparatus according to any preceding claim, comprising means for determining, in the sample obtained from the test subject, the concentration of SEQ ID No: 3, or a variant or fragment thereof, optionally wherein the means for determining, in the sample obtained from the test subject, the concentration of SEQ ID No: 3, or a variant or fragment thereof comprises an anti-T14 antibody or antigen-binding fragment thereof.

10. A method or apparatus according to any preceding claim, wherein the sample is nasal discharge or fluid, saliva, blood, venous blood, arterial blood, blood plasma, blood serum, capillary blood, non-venous blood, non-arterial blood, pin-prick blood, spinal fluid, urine, sweat, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof.

11. A method or apparatus according to claim 10, wherein the sample comprises nasal discharge or nasal fluid.

12. A method or apparatus according to claim 10, wherein the sample comprises saliva.

13. A method or apparatus according to claim 10, wherein the sample comprises capillary blood or pin-prick blood.

14. A method or apparatus according to any preceding claim, comprising an inhibition lateral flow test.

15. A method or apparatus according to any preceding claim, comprising a sandwich lateral flow test.

16. A method or apparatus according to any preceding claim, wherein the apparatus comprises an LFT cartridge, which comprises a sample pad onto which the sample is placed, and wherein the LFT cartridge comprises anti-T14 antibodies, which are optionally labelled with a labelling moiety, such as a gold nanoparticle.

17. A method or apparatus according to claim 16, wherein the LFT cartridge comprises a fixed T14 peptide test line comprising T14 peptide attached thereto and/or wherein the LFT cartridge comprises a control line comprising an anti-species labelled antibody, which is not specific for T14.

18. A method or apparatus according to claim 17, wherein when the sample comprises T14 peptide, the peptide is captured by the anti-T14 antibody creating a conjugate of T14-antibody, which T14-antibody conjugate is unable to bind to the fixed T14 peptide that is attached to the test line, and wherein anti-T14 antibody that is not bound to sample T14 peptide in the sample flows and binds to the anti-species labelled antibody attached to the control line.

19. A method or apparatus according to any one of claims 14-18, wherein when the sample does not comprise T14 peptide, no conjugate of T14-antibody is created, and unbound anti-T14 antibody flows and binds to the LFT fixed T14 peptide that is attached to the test line, and to the anti-species antibody control attached to the control line.

20. A method or apparatus according to any one of claims 15-19, wherein the LFT cartridge comprises a first anti-T14 antibody, which binds to the first epitope on the T14 peptide, and the LFT cartridge comprises a T14 peptide test line comprising a second anti-T14 antibody, which binds to a second epitope on the T14 peptide.

21. A method or apparatus according to claim 20, wherein the LFT cartridge comprises a control line comprising an anti-species labelled antibody, which is not specific for T14.

22. A method or apparatus according to claim 20 or 21, wherein when the sample comprises T14 peptide, it is captured by the first anti-T14 antibody thereby creating a conjugate of T14-antibody, which conjugate is itself captured by the second anti-T14 antibody which is attached to the cartridge along the test line, and any additional anti-T14 antibody that has not bound to T14 peptide in the sample flows and binds to the anti-species control affixed to the control line.

23. A method or apparatus according to any one of claims 20-22, wherein when the sample does not comprise T14 peptide, no conjugate of T14-antibody is created, and unbound first anti-T14 antibody cannot bind to the second T14 antibody, wherein the first anti-T14 antibody flows to and binds the anti-species control affixed to the control line.

24. A method or apparatus according to any preceding claim, wherein the method or apparatus is qualitative, semi-quantitative or quantitative.

25. A method or apparatus according to any one of claims 16-24, comprising means for quantitatively measuring the results of the LFT cartridge, optionally wherein the lateral flow cartridge is adapted to be connected to a reader, computer, tablet or smartphone, which runs software for detecting the test line and control line and, based on the concentrations/values of the test line and control line, calculates the concentration of the peptide of SEQ ID No: 3, or a variant or fragment thereof in the sample.

26. A method of diagnosing or prognosing a neurodegenerative disorder in a subject, the method comprising detecting, in a nasal discharge, nasal fluid, or a pin-prick blood sample obtained from a test subject, a peptide comprising or consisting of SEQ ID No: 3 (T14), or a variant or fragment thereof, wherein detecting the presence of the peptide comprising or consisting of SEQ ID No: 3, or a variant or fragment thereof in the sample indicates that the test subject has neurodegenerative disorder, or a pre-disposition thereto or a negative prognosis thereof.

27. A neurodegenerative disorder diagnostic or prognostic apparatus, for diagnosing or prognosing neurodegenerative disorder in a subject, the apparatus comprising means for detecting, in a nasal discharge, nasal fluid, or a pin-prick blood sample obtained from a test subject, a peptide comprising or consisting of SEQ ID No: 3 (T14), or a variant or fragment thereof, wherein detection of the peptide comprising or consisting of SEQ ID No: 3 (T14), or a variant or fragment thereof corresponds to the subject having a neurodegenerative disorder, or a pre-disposition thereto or a negative prognosis thereof.

28. A method or apparatus according to either claim 26 or claim 27, comprising an assay adapted to detect the presence and/or absence of the peptide of SEQ ID No: 3, or a variant or fragment thereof in the sample.

29. A method or apparatus according to claim 28, wherein the assay comprises lateral flow, an immunoassay, a non-immuno-based assay, Western Blot analysis, enzyme-linked immunosorbent assay (ELISA), fluorometric assay, chemiluminescent assay, or radioimmunoassay analyses.

30. A method or apparatus according to claim 29, wherein the assay comprises lateral flow.

31. A method or apparatus according to any one of claims 26-30, wherein the concentration of (i) a soluble peptide comprising or consisting of SEQ ID No: 3, or a variant or fragment thereof, and/or of (ii) an aggregated peptide comprising or consisting of SEQ ID No: 3, or a variant or fragment thereof, is determined.

32. A method or apparatus according to claim 31, wherein the apparatus comprises means for determining, in the sample obtained from the test subject, the concentration of SEQ ID No: 3, or a variant or fragment thereof, optionally wherein the means for determining, in the sample obtained from the test subject, the concentration of SEQ ID No: 3, or a variant or fragment thereof comprises an anti-T14 antibody or antigen-binding fragment thereof.

33. A method or apparatus according to any one of claims 9-25, or claim 32, wherein the antibody or antigen-binding fragment thereof specifically binds to SEQ ID No: 3, optionally one or more amino acid in SEQ ID No: 11, preferably wherein the antibody or antigen-binding fragment thereof does not bind to SEQ ID No: 2 (i.e. T30), SEQ ID No: 13 (i.e. T15) and/or SEQ ID No: 14 (i.e. Aβ).

34. A method or apparatus according to any preceding claim, wherein the concentration of SEQ ID No: 3, or a variant or fragment thereof, in the sample is:
(i) between 0.1 and 1000 ng, or between 0.2 and 750 ng, or between 0.5 and 500 ng per mg protein in the sample;
(ii) between 1 and 400 ng, or between 2 and 300 ng, or between 3 and 200 ng per mg protein in the sample; and/or
(iii) between 4 and 100 ng, or between 5 and 75 ng, between 10 and 50 ng, or between 20 and 40 ng per mg protein in the sample.

35. A method or apparatus according to any preceding claim, wherein the subject has, or is suspected of having, a neurodegenerative disease selected from a group consisting of: Alzheimer's disease; Parkinson's disease; Huntington's disease; Motor Neurone disease; Spinocerebellar type 1, type 2, and type 3; Amyotrophic Lateral Sclerosis (ALS); schizophrenia; Lewy-body dementia; and Frontotemporal Dementia.

36. A method or apparatus according to any preceding claim, wherein the subject has, or is suspected of having, Alzheimer's Disease.

37. A method or apparatus according to any preceding claim, wherein the methods and apparatus are used on a subject:
(i) who is already diagnosed with a neurodegenerative disorder;
(ii) who is symptomatic and showing signs of cognitive decline or dementia; and/or
(iii) who is not suspected of having a neurodegenerative disorder, and is pre-symptomatic.

38. A method or apparatus according to any preceding claim, comprising detection of soluble and/or aggregated peptide comprising or consisting of one or more of any of T7-T13 (i.e. SEQ ID No: 4-10).

39. A method or apparatus according to any preceding claim, comprising determining the Braak stage of the subject.

40. A method or apparatus according to claim 39, wherein the subject is any of Braak stage I, II or III, preferably Braak stage I or II.

41. A method or apparatus according to either claim 39 or 40, comprising:
(a) analysing, in the sample obtained from the test subject, the concentration of the peptide comprising or consisting of SEQ ID No: 3 (T14), or a variant or fragment thereof; and
(b) comparing this concentration with a reference value from a control population of deceased subjects having known Braak stages for concentrations of soluble peptide comprising or consisting of SEQ ID No: 3, or a variant or fragment thereof,
wherein the Braak stage of the living test subject is determined by comparing the concentration of the peptide comprising or consisting of SEQ ID No: 3, or a variant or fragment thereof, with the respective reference value that is associated with a Braak stage.

42. A method or apparatus according to claim 41, wherein soluble SEQ ID No: 3 (T14), or a variant or fragment thereof is analysed.

43. A method or apparatus according to claim 42, wherein:
(i) a lower concentration of soluble peptide comprising or consisting of SEQ ID No: 3, or a variant or fragment thereof, compared to the reference value is indicative of Braak stage I;
(ii) a lower concentration of soluble peptide comprising or consisting of SEQ ID No: 3, or a variant or fragment thereof, compared to the reference value is indicative of Braak stage II; and/or
(iii) a lower concentration of soluble peptide comprising or consisting of SEQ ID No: 3, or a variant or fragment thereof, compared to the reference value is indicative of Braak stage III.

44. A method or apparatus according to any preceding claim, comprising measuring the rate of cognitive decline by a Mini Mental State Examination (MMSE) score and/or a Preclinical Alzheimer Cognitive Composite (PACC) score.

45. A method or apparatus according to any preceding claim, comprising the use of lateral flow to detect of SEQ ID No: 3, or a variant or fragment thereof, in nasal discharge, nasal fluid or saliva, to diagnose or prognose pre-symptomatic Alzheimer's Disease.
